SEQUENCE LISTING (3 sequences)

SEQ ID NO: 1 - DNA, 15373 bp, Porcine reproductive and respiratory syndrome virus (JX143)
atgacgtataggtgttggctctatgccacggcatttgtattgtcaggagctgtgaccattggcacagcccaaaacttgctgcacgggaacaccctcctgtgacagccctcttcagggggattaggggtctgtccctaacaccttgcttccggagttgcactgttttacggtctctccacccctttaaccatgtctgggatacttgatcggtgcacgtgtacccccaatgctagggtgtttgtggcggagggccaggtctactgcacacgatgtctcagtgcacggtctctccttcctctgaatctccaagttcctgagcttggggtgctgggtctattttataggcccgaagagccactccggtggacgttgccacgtgcattccccactgtcgagtgctcccccgccggggcctgttggctttctgcgatttttccgattgcacgaatgactagtggaaacctgaactttcaacaaagaatggtgcgggtcgcagctgaaatctacagacccggccaactcacccctacagttctaaagactctacaagtttatgaacggggttgtcgctggtaccccattgtcgggcccgtccctggggtgggcgtttacgccaactccctgcatgtgagtgacaaacctttcccgggagcaactcatgtgttaaccaacttgccgctcccgcagaggcccaaacctgaggacttttgcccttttgagtgtgctatggctgacgtctatgacattggtcgtggcgctgtcatgtatgtggccggaggaaaggtctcttgggcccctcgtggtgggaatgaagtgaaatttgaacctgttcccaaggagttgaagttggttgcgaaccgactccacacctccttcccgccccatcacgtagtggacatgtccgagtttaccttcatgacccctgggagtggtgtctccatgcgggttgagtaccaatacggctgcctccctgctgacactgtccctgaaggaaactgctggtggcgcttgtttgactcgctcccaccggaagttcagtacaaagaaattcgccatgctaaccaatttggctatcaaaccaagcatggtgtccctggcaagtacctacagcggaggctgcaagttaatggtcttcgggcagtgaccgacacacatggacctatcgtcatacagtacttctctgttaaggagagttggatccgccacctgaagttggtggaagaacccagcctccccgggtttgaggatctcctcagaatcagggttgagcccaatacgtcaccactggctagaaaggatgagaagattttccggtttggcagtcataagtggtacggtgccggaaagagagcaaggaaaacacgctctggtgcgactactatggtcgctcatcacgcttcgtccgctcatgaaacccggcaggccacgaagcacgagggtgccggcgctaacaaggccgagcatctcaagcgctactctccgcctgccgaagggaactgtggttggcactgcatttccgccatcgccaaccggatggtgaattccaactttgagaccacccttcctgaaagggtaaggccttcagatgactgggccactgacgaggatcttgtgaacaccatccaaatcctcaggctccctgcggccttggacaggaacggcgcttgcggtagcgccaagtacgtgcttaaactggagggtgagcattggactgtctctgtgatccctgggatgtcccctactttgctcccccttgaatgtgttcagggttgttgtgagcataagggcggtcttgtttccccggatgcggtcgaaatttccggatttgatcctgcctgccttgaccgactggctaaggtaatgcacttgcctagcagtaccatcccagccgctctggccgaattgtccgacgactccaaccgtccggtttccccggccgctactacgtggactgtttcgcaattctatgctcgtcatagaggaggagatcatcatgaccaggtgtgcttagggaaaatcatcagcctttgtcaagttattgaggattgctgctgccatcagaataaaaccaaccgggctactccggaagaggtcgcggcaaagattgatcagtacctccgtggcgcaacaagtcttgaggaatgcttggccaaacttgagagagtttccccgccgagcgctgcggacacctcctttgattggaatgttgtgcttcctggggttgaggcggcgaatcagacaaccgaacaacctcacgtcaactcatgctgcaccctggtccctcccgtgactcaagagcctttgggcaaggactcggtccctctgaccgccttctcactgtccaattgctattaccctgcacaaggtgacgaggttcatcaccgtgagaggttaaattccgtactctctaagttggaagaggttgtcctggaagaatatgggctcatgtccactggacttggcccgcgacccgtgctgccgagcgggctcgacgagcttaaagaccagatggaggaggatctgctaaaactagccaacacccaggcgacttcagaaatgatggcctgggcagctgagcaggtcgatttaaaagcttgggtcaaaagctacccgcggtggacacctccaccccctccaccaagagttcaacctcgcagaacaaagtctgtcaaaagcttgccagaggacaagcctgtccctgctccgcgcaggaaggtcagatccgattgcggcagcccggttttgatgggcgacaatgtccctaacggttcggaagaaactgtcggtggtcccctcaattttccgacaccatccgagccgatgacacctatgagtgagcccgtacttgtgcccgcgtcgcgacgtgtccccaagctgatgacacctttgagtgggtcggcaccagttcctgcaccgcgtagaactgtgacaacaacgctgacgcaccaggatgagcctctggatttgtctgcgtcctcacagacggaatatgaggctttccccctagcaccgtcgcagaacatgggcatcctggaggcgggggggcaagaagctgaggaagtcctgagtgaaatctcggatatactaaatgacaccaaccctgcacctgtgtcatcaagcagctccctgtcaagtgttaagatcacacgcccaaaatactcagctcaagccatcatcgactctggcgggctttgcagtgggcatctccaaaaggaaaaagaagcatgcctcagcatcatgcgtgaggcttgtgatgcgtccaagcttagtgatcctgctacgcaggagtggctctctcgcatgtgggatagggttgacatgctgacttggcgcaacacgtctgcttaccaggcgtttcgcatcttaaatggcaggtttgagtttctcccaaagatgattctcgagacaccgccgccccacccgtgcgggtttgtgatgttacctcacacgcctgcaccttccgtgagtgcagagagtgatctcaccattggttcagtggccaccgaggatgttccacgcatcctcgggaaaataggagacactgacgagctgcttgaccggggtccctcggcaccctccaagggagaaccggtctgtgaccaacctgccaaagatccccggatgtcgccgcgggagtctgacgagagcataatagctccgcccgcagatacaggtggtgtcggctcattcactgatttgccgtcttcagatggtgtggatgtggacgggggggggccgttaagaacggtaaaaacaaaagcaggaaggctcttagaccaactgagctgccaggtttttagcctcgtttcccatctccctattttcttctcacacctcttcaaatctgacagtggttattctccgggtgattggggttttgcagcttttactctattttgcctctttctatgttacagttacccattcttcggttttgctcccctcttgggtgtattttctgggtcttctcggcgtgtgcgaatgggggtttttggctgctggttggcttttgctgttggtctgttcaagcctgtgtccgacccagtcggcactgcttgtgagtttgactcgccagagtgtaggaacgtccttcattcttttgagcttctcaaaccttgggaccctgtccgcagccttgttgtgggccccgtcggtctcggccttgccattcttggcaggttattgggcggggcacgctacatctggcactttttgcttaggcttggcattgttgcagactgtatcttggctggagcttatgtgctttctcaaggtaggtgtaaaaagtgctggggatcttgtgtaagaactgctcctaatgagatcgccttcaacgtgttcccttttacacgtgcgaccaggtcgtcactcatcgacctgtgcgatcggttttgcgcaccaaaaggcatggaccccatttttctcgccactgggtggcgtgggtgctggaccggccggagtcccattgagcaaccttctgaaaaacccatcgcgttcgcccagctggatgagaagaggattacggctagaactgtggtcgctcagccttatgatcccaaccaggccgtaaagtgcttgcgggtattacaggcgggtggggcgatggtggccgaggcagtcccaaaagtggtcaaagtttccgctattccattccgagctcctttctttcccgctggagtgaaagttgatcctgagtgcagaatcgtggttgatcccgatacttttactacagccctccggtctggctattccaccgcgaacctcgtccttggtacgggggactttgcccagctgaatggactaaagatcaggcaaatttccaagccttcagggggaggcccacacctcattgctgccttgcatgttgcctgctcgatggcgttacacatgcttgctggtgtttatgtaactgcagtggggtcctgcggtaccggtaccaacgatccgtggtgcactaacccgtttgccgtccctggctacggacctggctctctttgcacgtctagattgtgcatctcccaacacggcctcaccttgcccttgacagcacttgtggcgggattcggccttcaagagattgccttggtcgttttgatttttgtctccatcggaggcatggctcataggttgagttgtaaggctgacatgttgtgcatcttactcgcaatcgctagttatgtttgggtacctcttacctggttgctttgtgtgtttccttgttggttgcgctgtttctctttgcaccccctcaccatcctatggttggtgtttttcttgatttctgtaaatataccctcaggagtcttggccgtggtgttgttgatttctctctggcttttaggtcgttatactaacgttgctggtcttgtcactccctatgacattcatcattacaccagtggcccccgcggtgttgccgccttggctaccgcaccagatgggacctacttggccgctgtccgccgcgctgcgctgactggtcgtaccatgctgttcaccccgtctcagctcgggtccctccttgagggcgctttcagaactcaaaagccctcactgaacaccgtcaatgtggtcgggtcctccatgggctctggcggagtgttcactattgacgggaaaatcaagtgcgtgactgccgcacatgtccttacgggtaactcagctagggtttccggggtcggcttcaatcaaatgcttgactttgatgtaaaaggggacttcgccatagctgattgcccgaattggcaaggggttgctcccaaggcccagttctgcgaggatgggtggactggtcgcgcctattggctgacatcctctggcgttgaacccggtgttattgggaatgggttcgccttctgcttcaccgcgtgtggcgattctggatccccagtgattaccgaagccggtgagcttgtcggcgttcacacaggatcaaacaaacaaggaggaggcattgtcacgcgcccctcaggccagttttgtaatgtgaagcccatcaagctgagcgagttgagtgaattcttcgctggacctaaggtcccgctcggtgatgtgaaaattggcagtcacataattaaagacacatgcgaggtgccttcagatctttgtgccctgcttgctgccaaacccgaactggaaggaggcctttccacagttcaacttctgtgtgtgtttttcctcctgtggagaatgatggggcatgcttggacgcccttggttgctgtggggtttttcatcctgaatgagattctcccagctgtcctggtccggagtgttttctcctttgggatgtttgtgctatcttggctcacaccatggtctgcgcaagtcctgatgatcaggcttctgacagcagcccttaacagaaacagatggtctcttggtttttacagccttggtgcagtaaccagttttgtcgcagatcttgcggtaactcaagggcatccgttacaggtggtaatgaacttaagcacctatgccttcctgccccggatgatggttgtgacctcgccagtcccagtgatcgcgtgtggtgttgtgcacctccttgccataattttgtacttgtttaagtaccgctgccttcacaatgtccttgttggcgatggggtgttctcttcggctttcttcttgcgatactttgccgagggaaagttgagggaaggggtgtcgcaatcctgcgggatgagtcatgagtcgctgactggtgccctcgccatgagactcactgacgaggacttggatttccttacgaaatggactgattttaagtgctttgtttctgcgtccaacatgaggaatgcagcgggccaatttatcgaggctgcttatgcaaaagcactaagaattgaacttgctcagttggtacaggttgataaggtccgaggtaccatggccaaactcgaggcttttgccgataccgtggcaccccaactctcgcccggtgacattgttgttgcccttggccacacgcctgttggcagcatcttcgacctaaaggttggtagcaccaagcatactctccaagccattgagactagagtccttgccgggtccaaaatgactgtggcgcgtgtcgttgacccaacccccgcacccccacccgtacctgtgcccatccctctcccaccgaaagttctggagaacggtcccaatgcctggggggatgaggaccgtttgaacaagaagaagaggcgcaggatggaagccgtcggcatttttgtcatggacgggaagaagtaccagaaattttgggacaagaattccggtgatgtgttttatgaggaggtccatattagcacagacgagtgggagtgccttagaactggcgaccctgtcgactttgatcctgagacagggattcagtgtgggcatatcaccattgaagacaaggtttacaatgtcttcacctccccatctggtaggagattcttggtccccgccaaccccgagaatagaagagctcagtgggaagccgccaagctttccgtggagcaagcccttggcatgatgaacgtcgacggcgaactgactgccaaagaactggagaaactgaaaagaataattgacaaactccagggcctgactaaggagcagtgtttaaactgctagccgccagcggcttgacccgctgtggtcgcggcggcttagttgttactgagacagcggtaaaaatagtcaaatttcacaaccggaccttcaccctaggacctgtgaacttaaaagtggccagtgaggttgagctaaaagacgcggttgagcacaaccaacatccggttgccagaccggttgatggtggtgttgtgctcctgcgctctgcagttccttcgcttatagatgtcttgatctccggcgctgatgcatctcctaagttactcgcccgccacgggccgggaaacactgggattgatggcacgctttgggattttgaggccgaggctactaaagaggaagttgcactcagtgcgcaaataatacaggcttgtgatattaggcgcggcgacgcgcctgaaattggtctcccttataagttgtaccctgttaggggcaaccctgagcgggtaaaaggagttttacagaatacaaggtttggagacataccttacaaaacccccagtgacactggaagcccggtgcacgcggctgcctgcctcacgcctaatgctactccggtgactgatgggcgctccgtcttggctacaaccatgccctctggctttgagttgtatgtgccgaccattccagcgtccgtccttgattatcttgattctaggcctgactgccctaaacagttaacagagcacggttgtgaggatgctgcattaagagacctctccaagtatgatttgtccacccaaggctttgttttgcctggagttcttcgcctcgtgcggaagtacctgttcgcccacgtgggtaagtgcccgcccgttcatcggccttccacttaccctgctaagaattctatggctggaataaatgggaacaggtttccaaccaaggacattcagagcgtccctgaaatcgacgttctgtgcgcacaggctgtgcgagaaaactggcaaactgttaccccttgtaccctcaagaaacagtactgtgggaagaagaagactaggacaatacttggcaccaataacttcattgcgttggcccatcgggcagcgttgagtggtgttacccagggcttcatgaaaaaagcgttcaactcgcccatcgccctcgggaaaaacaaatttaaggagctacaagccccggtcctaggcaggtgccttgaagctgatcttgcgtcctgcgatcgatccacacctgcaattgtccgctggtttgccgccaatcttctttatgaactcgcctgtgctgaggagcatctaccgtcgtacgtgctgaactgctgccacgacttactggtcacgcagtccggcgcggtgactaagaagggtggcctgtcgtctggcgacccgattacctctgtgtcaaacaccatttacagcttagtgatatatgcacagcacatggtgctcagttacttcaaaagtggtcaccctcatggccttctgtttctgcaagaccagctaaagtttgaggacatgctcaaggttcaacccctgatcgtctattcggacgaccttgtgctgtatgccgagtctccctccatgccaaactaccactggtgggttgaacatctgaatcttatgctgggtttccagacggacccaaagaagacaaccatcacagactcaccatcattcctaggttgcaggataataaatgggcgccagctagtccctaaccgtgacaggatcctcgcggccctcgcctaccacatgaaggcaagtaatgtttctgaatactacgcctcggcggctgcaatactcatggacagctgtgcttgtttagagtatgatcctgaatggtttgaagagctcgtggttgggatggcgcagtgcgcccgcaaggacggctacagctttcctggcccaccgttcttcttgtccatgtgggaaaaactcaggtccaatcatgaggggaagaagtccagaatgtgcgggtactgcggggccccggctccgtacgccactgcctgtggtctcgatgtctgtgtttaccacacccacttccaccagcattgtcctgttataatctggtgtggccacccggcgggttctggttcttgtagtgagtgcgaaccccccctaggaaaaggcacaagccctctagatgaggtgttagaacaagttccgtacaagcctccgcggactgtgatcatgcatgtggagcagggtctcacccctcttgacccaggtagataccagactcgccgcggattagtctccgttaggcgtggcattaggggaaatgaagtcgacctaccagacggtgattacgctagcaccgccttgctccctacttgtaaagagatcaacatggtcgctgtcgcctctaacgtgttgcgcagcaggtttatcatcggcccacccggtgctgggaaaacacactggcttcttcaacaagtccaggatggtgatgtcatttacacgccaactcaccagaccatgctcgacatgattagggctttggggacgtgccggttcaacgttccagcaggtacaacgctgcaattccctgccccctcccgtaccggcccatgggttcgcatcttggccggcggttggtgtcctggcaagaactccttcctggatgaagcggcgtattgcaatcaccttgatgtcttgaggcttctcagtaaaacaactctcacttgcctaggagacttcaaacaactccaccctgtgggttttgactcccattgctatgtatttgacatcatgcctcagacccaattaaagaccatctggaggttcgggcagaatatctgtgatgccattcaaccagattacagggacaaacttatgtccatggtcaacacgacccgtgtgacctacgtggaaaaacctgtcaggtatgggcaagtcctcaccccctaccacagggaccgagaggacggcgccattactatcgactccagtcaaggcgccacatttgatgtggttacactgcatttgcccactaaagattcactcaacaggcaaagagctcttgttgctatcaccagggcaagacatgctatcttcgtgtatgacccacacaggcaattgcagagcatgtttgatcttcccgcgaaaggcacacccgtcaacctcgcagtgcaccgtgacgaacagctgatcgtattagacagaaacaacagagaaatcacggttgctcaggctctaggcaatggagataaattcagggccacagataagcgcgttgtagattctctccgcgctatttgcgcagacctggaagggtcgagctccccgctccccaaggtcgcgcataacttgggattctatttctcacctgatttgactcagtttgctaaactcccggcagaacttgcgccccactggcccgtggtgacaacccagaacaatgaaaggtggccagatcggctggtagccagccttcgccctatccataaatatagccgcgcgtgcattggtgccggctatatggtgggcccctcggtgtttttaggcacccctggggttgtgtcatactatctcacaaaatttgttagaggcgaggctcaagtgcttccggagacagtcttcagcaccggccgaattgaggtagattgtcgagagtatcttgatgatcgggagcgagaagttgctgagtccctcccacatgccttcatcggcgatgtcaaaggtaccaccgttgggggatgtcatcacgttacctccaaataccttccgcgcttccttcccaaggaatcagttgcggtggtcggggtttcgagccccgggaaagccgcgaaagcagtttgcacattgacggatgtgtacctcccagaccttgaagcgtacctccacccagagacccagtccaggtgctggaaagtgatgttggactttaaggaggttcgactgatggtatggaaagacaagacggcctattttcaacttgaaggccgccattttacctggtatcaacttgcaagctacgcctcatacatccgagttcctgttaattctactgtgtacttggacccctgcatgggccctgctctttgcaacagaagggttgtcgggtccacccattggggagctgacctcgcagtcaccccttatgattacggtgccaaaattattctgtctagtgcataccatggtgaaatgcctccaggttacaaaattctggcgtgcgcggagttctcgcttgatgacccagtaaggtacaaacacacctggggatttgaatcggatacagcgtatctgtacgagtttactggaaatggtgaggactgggaggattacaatgatgcgtttcgggcgcgccagaaagggaaaatttataaagctaatgccaccagcatgaggtttcattttcccccgggccctgtcattgaaccaactttaggcctgaattgaaatgaaatggggtctatgcaaagcctctttgacaaaattggccaactttttgtggatgctttcacggaatttctggtgtccattgttgatatcatcatatttttggccattttgtttggcttcacaatcgccggttggctggtggtcttatgcatcagactggtttgctccgcggtactccgtgcgcgctctaccgttcaccctgagcaattacagaagatcttatgaggcctttctttctcagtgtcaggtggacattcccacctggggcgtcaaacaccctttgggggtgctttggcaccataaggtgtcaaccctgattgatgaaatggtgtcgcgtcgaatgtaccgcatcatggaaaaagcagggcaggctgcctggaaacaggtggtgagcgaggctacattgtctcgcattagtggtttggatgtggtggctcactttcaacatcttgccgctattgaagccgagacttgtaaatatttggcctcccggctacccatgctgcacaacctgcgcttgacagggtcaaatgtaaccatagtgtataatagtactttggatcaggtgtttgccattttcccaacccctggttcccggccaaagcttcatgattttcagcaatggctaatagctgtacattcctccatattttcttccgttgcagcttcttgtactctttttgttgtgctgtggttgcgaattccaatgctacgttctgtttttggtttccgctggttaggggcaacttttcttttgaactcatggtgaattacacggtatgcccgctttgcccaacccggcaggcagccgctgagatccttgaacccggcaagtctttttggtgcaggatagggcatgaccgatgtagtgagaacgatcatgacgaactagggttcatggttccgcctggcctttccagcgaaggccacttgaccagtgtttacgcctggttggcgttcctgtccttcagctacacggcccagttccatcccgagatatttgggatagggaatgtgagtcaagtttatgttgacatcaagcaccaattcatctgcgctgttcacgacggggataacgccaccttgcctcgccatgacaatatttcagccgtatttcagacctactaccaacaccaggtcgacggcggcaattggtttcacctggaatggctgcgccctttcttttcctcttggttggttttaaatgtttcgtggtttctcaggcgttcgcctgcaaaccatgtttcagttcgagtctttcggacatcaaaaccaacaccaccgcagcatcagacttcgttgtcctccaggacatcagctgccttaggcatggcgactcgtcctctccgacgattcgcaaaagttctcagtgccgcacggcgatagggacgcccgtgtacatcaccatcactgccaatgtcacagatgaaaattatctacattcttctgatctcctcatgctttcttcttgccttttctatgcttccgagatgagtgaaaagggattcaaagtggtgtttggcaatgtgtcaggcatcgtggctgtgtgcgtcaactttaccagctacgtccaacacgtcaaggagtttacccaacgctccttagtggtcgatcatgtgcgactgcttcatttcatgacacctgagaccatgaggtgggcaaccgttttagcctgtctttttgccatcctactggcaatttgaatgtttaagtatgttggggaagtgcttgaccgcgtgctgttgctcgcgattgctttttttgtggtgtatcgtgccgttctatcttgctgtgctcgtcaacgccagcaacaacaacagctctcatattcagttgatttataacttaacgctatgtgagctgaatggcacagattggctggcacaaaaatttgactgggcagtggagacttttgtcatcttccccgtgttgactcacattgtttcctatggggcactcaccaccagccatttccttgacacagttggtctggccactgtgtccaccgccggatattatcacgggcggtatgtcttgagtagcatttacgcagtctgtgctctggctgcgctgatttgctttgtcattaggcttgcgaagaactgcatgtcctggcgctactcttgtaccagatataccaacttccttctggacactaagggcagactctatcgttggcggtcgcccgtcattgtggagaaagggggtaaggttgaggtcgaaggtcacctgatcgacctcaagagagttgtgcttgatggttccgcggcaacccctttaaccagagtttcagcggaacaatggggtcgtctctagacgacttctgcaatgatagcacagctccacagaaggtgcttttggcgttttccattacctacacgccagtgatgatatatgctctaaaggtaagtcgcggccgactgctagggcttctgcaccttttgatctttctgaattgtgcttttaccttcgggtacatgacattcgtgcactttgagagcacaaatagggtcgcgctcactatgggagcagtagttgcacttctttggggagtgtactcagccatagaaacctggaaattcatcacctccagatgccgtttgtgcttgctaggccgcaagtacattctggcccctgcccaccacgtcgaaagtgccgcgggctttcatccgattgcggcaaatgataaccacgcatttgtcgtccggcgtcccggctccactacggtcaacggcacattggtgcccgggttgaaaagcctcgtgttgggtggcagaaaagctgttaagcagggagtggtaaaccttgttaaatatgccaaataacaacggcaagcagcaaaagaaaaagaaggggaatggccagccagtcaatcagctgtgccaaatgctgggtaagatcatcgcccaacaaaaccagtccagaggcaagggaccggggaagaaaaataggaagaaaaacccggagaagccccatttccctctagcgactgaagatgacgtcaggcatcactttacccctagtgagcggcaattgtgtctgtcgtcgatccagactgccttcaatcagggcgctggaacttgtgccctgtcagattcagggaggataagttacactgtggagtttagtttgccgacgcaacatactgtgcgtctgatccgcgccacagcatcaccctcagcatgatgggctggcattctttggcacctcagtgttagaattgggagaatgtgtggtgaatggcactgattgacactgtgcctctaagtcacctattcaattagggcgaccgtgtgggggtaaagtttaattggcgagaaccatgcggccgtaattaaaaaaaaaaaaaaaaaaaaaaaaaaaaaaaaaaaaaaaaaaaaaaaaaaaaa

SEQ ID NO: 2 - protein, 1196 aa, Porcine reproductive and respiratory syndrome virus (JX143)
GAGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILETPPPYPCEFVMMPHTPAPSVGAESDLTIGSVATEDVPRILEKIENVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLGGARCIWHFLLRLGIVADCILAGAYVLSQGRCKKCWGSCIRTAPNEVAFNVFPFTRATRSSLIDLCDRFCAPKGMDPIFLATGWRGCWAGRSPIEQPSEKPIAFAQLDEKKITARTVVAQPYDPNQAVKCLRVLQSGGAMVAKAVPKVVKVSAVPFRAPFFPTGVKVDPDCRVVVDPDTFTAALRSGYSTTNLVLGVGDFAQLNGLKIRQISKPSG

SEQ ID NO: 3 - DNA, 603 bp, Porcine reproductive and respiratory syndrome virus (JX143)
atgttggagaaatgcttgaccgcgggctgttgctcgcgattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttag

(19) United States
(12) Patent Application Publication
(10) Pub. No.: US 2011/0117129 A1
(43) Pub. Date: May 19, 2011

(54) Vaccine Against Highly Pathogenic Porcine Reproductive and Respiratory Syndrome (HP PRRS)

(75) Inventors: Michael B. Roof, St. Joseph, MO (US); Eric Vaughn, St. Joseph, MO (US)

(73) Assignee: BOEHRINGER INGELHEIM VETMEDICA, INC., St. Joseph, MO (US)

(21) Appl. No.: 13/055,590

(22) PCT Filed: Aug. 24, 2009
(86) PCT No.: PCT/US09/54775

§ 371 Date: Jan. 24, 2011

Related U.S. Application Data
(60) Provisional application No. 61/091,614, filed on Aug. 25, 2008.

(51) Int. Cl. A61K 39/225; A61P 37/00

(52) U.S. Cl. 424/221.1

(57) ABSTRACT

The present invention is related to methods and attenuated viral compositions for use in preventing and treating a high fever disease forms associated with porcine reproductive and respiratory syndrome (PRRS), such as highly pathogenic porcine reproductive and respiratory syndrome (HP PRRS), a viral disease affecting swine.

### SEQUENCE LISTING

This application contains a sequence listing in paper format and in computer readable format, the teachings and content of which are hereby incorporated by reference.

### BACKGROUND OF THE INVENTION

1. Technical Field

The present invention is generally related to vaccines against infectious diseases. More particularly, it relates to vaccines against Highly Pathogenic Porcine Reproductive and Respiratory Syndrome (HP PRRS), a viral disease affecting swine.

2. Background Information

Porcine reproductive and respiratory syndrome (PRRS) is recognized as a serious swine disease and is characterized with either reproductive failure in pregnant sows, or respiratory tract distress particularly in sucking pigs. This viral disease was first discovered in the United States in 1987, subsequently found in Europe, and identified in Asia in the early 1990s. To date, PRRS has spread worldwide with the characteristics of endemic in those swine-cultivating countries, causing enormous economic losses each year. The etiological agent of PRRS is porcine reproductive and respiratory syndrome virus (PRRSV) which, together with lactate dehydrogenase-elevating virus of mice (LDEV), equine arteritis virus (EAV), and simian hemorrhagic fever virus (SHFV), belongs to the family Arteriviridae within the order Nidovirales.

PRRSV, a member of the small enveloped viruses, has a single-strand positive-sense RNA (+ssRNA) genome of approximately 15.1-15.5 kb, comprising at least 8 open reading frames (ORFs) that encode about 20 putative proteins. The genome also contains two untranslated regions (UTR) at both the 5′- and 3′-ends. In detail, ORF1 (ORF1a and ORF1b) is located downstream of the 5′-UTR and occupies more than two-thirds of the whole genome. ORF1a is translated directly, whereas ORF1b is translated by a ribosomal frameshift, yielding a large ORF1ab poly-protein that is proteolytically cleaved into products related to the virus transcription and replication machinery. ORFs 2-7, located upstream of the 3′-UTR, encode a series of viral structural proteins associated with the virion, such as the envelope protein (E) and nucleocapsid protein (N). These proteins are all translated from a 3′-coterminal nested set of sub-genomic mRNAs (sgmRNAs).

Phylogenetic analysis of PRRSV isolates from different geographical regions worldwide clearly indicates the existence of two major genotypes: Type I representing the European prototype (Lelystad virus, LV), and Type II representing the Northern American strain ATCC VR2332 (for the genomic sequence of VR2332, see GenBank accession No. AY150564) as a prototype (Murtaugh et al., Arch Virol. 1995; 140:1451-1460). Moreover, some studies have shown that ORF5 and the non-structural protein 2 (NSP2)-coding gene (nsp2) may represent the most genetically variable regions in PRRSV genomes. See SwissProt accession No. Q9WJB2 or SEQ ID NO:2 for the sequence of NSP2 of VR2332. It is also well documented that PRRSV strains differ greatly in their pathogenicities.

In 2006, an unparalleled large-scale outbreak of an originally unknown, but so-called “high fever” disease with symptoms of PRRS occurred, which spread to more than 10 provinces and affected over 2,000,000 pigs with about 400,000 fatal cases. Different from the typical PRRS, numerous adult sows were also infected by the “high fever” disease. This atypical PRRS pandemic was initially identified as a hog cholera-like disease manifesting neurological symptoms (e.g., shivering), high fever (40-42° C.), erythematous blanching rash, etc. Autopsies combined with immunological analyses clearly showed that multiple organs were infected by highly pathogenic PRRSVs with severe pathological changes observed (Tian et al., PLoS ONE. 2007; 2(6): e526). Whole-genome analysis of the isolated viruses revealed that these PRRSV isolates are grouped into Type II and are highly homologous to HB-1, a Chinese strain of PRRSV (96.5% nucleotide identity), and JX143 (Yuan et al, 2007 International PRRS Symposium, Chicago). For the genomic sequence of JX143, see SEQ ID NO:1, or EMBL/GenBank accession No. EU708726. It was furthermore observed that these viral isolates comprise a unique molecular hallmark, namely a discontinuous deletion of 30 amino acids in nonstructural protein 2 (NSP2) (Tian et al., PLoS ONE. 2007; 2(6): e526). The “high fever disease” form of PRRS is now also referred to as “highly pathogenic PRRS”, or HP PRRS.

Isolation of PRRS virus (PRRSV) and manufacture of vaccines against PRRS, either comprising modified live (attenuated) or inactivated PRRSV, have been described in a number of publications (WO 92/21375, WO 93/06211, WO93/03760, WO 93/07898, WO 96/36356). In particular, WO 93/03760 discloses methods of PRRS virus isolation, cultivation, attenuation, as well as manufacture of respective vaccines, and in particular the PRRS Type II prototype isolate ATCC VR-2332. WO 96/36356 discloses a particularly useful attenuated descendant of the aforementioned isolate, obtained by serial passaging in simian cells, which has been deposited under the accession number ATCC VR-2495. A respective modified live (MLV) vaccine product is commercially available from Boehringer Ingelheim under the brand Ingelvac® PRRS MLV. Another MLV vaccine based on a Type II isolate is commercially available under the brand Ingelvac® PRRS ATP.

An appropriate strategy in the prevention of PRRS is vaccination. However, it has been hitherto unknown whether vaccination would be effective against HP PRRS, and what type of vaccine could be used.

### DESCRIPTION OF THE INVENTION

The inventors have made the surprising discovery that attenuated strains of PRRS Type II virus may be used to vaccinate and protect swine from the effects of high fever disease forms associated with porcine reproductive and respiratory syndrome. The identification of prophylactic characteristics of attenuated strains of PRRS Type II viruses may allow for the treatment of pigs at high risk, for example, for HP PRRS. Such a vaccination or treatment program may help to reduce the probability or impact of other HP PRRS outbreaks similar to those that devastated the swine industry in China in 2006 and resulted in the culling of roughly 20 million pigs.

One aspect of the present invention provided herein includes a method of prophylactically protecting swine from the effects of a high fever disease comprising administering to a pig in need thereof an immunogenic composition comprising an effective amount of an PRRS Type II virus, preferably an attenuated PRRS Type II virus. The composition may further comprise a pharmaceutically acceptable carrier. The composition may still further comprise an adjuvant. The method may be used as a prevention or treatment measure. Moreover, the administration of an effective amount of such an immunogenic composition results in a lessening of the incidence of or severity of clinical signs of high fever disease forms of PRRS.

Also provided herein is a method for vaccinating swine against a high fever disease comprising administering to a pig an immunogenic composition comprising an effective amount of an PRRS Type II virus, preferably an attenuated PRRS Type II virus. The composition may further comprise a pharmaceutically acceptable carrier. The composition may still further comprise an adjuvant. Such a vaccination with an effective amount of the immunogenic composition will preferably result in a lessening of the incidence of or severity of clinical signs of high fever disease forms of PRRS.

The high fever disease may be a form that is associated with porcine reproductive and respiratory syndrome. Porcine reproductive and respiratory syndrome may be highly pathogenic (“HP PRRS”). HP PRRS or a high fever disease form may be detected in swine showing clinical signs of one or more of the following: rubefaction, blood spots, petechiae, erythematous blanching rashes, and pimples, frequently observed in ears, mouth, noses, back, and inner thigh. Other common symptoms may include high fever (greater than 40° C.), depression, anorexia, cough, asthma, lameness, shivering, disorder in the respiratory tract, and diarrhea. The HP PRRS is caused by a HP PRRS virus.

Another aspect of the present invention provided herein includes a method of prophylactically protecting swine from infection with HP PRRS comprising administering to a pig in need thereof an immunogenic composition comprising an effective amount of an PRRS Type II virus, preferably an attenuated PRRS Type II virus.

HP PRRS virus that became evident in 2002 in China as member of the PRRS type 2 genotype is correlated with the so-called high fever disease. HP PRRS virus thereafter became dominant in several Chinese provinces indicating a selective advantage in spreading within affected pig populations compared to other PRRS viruses.

The term “HP PRRS virus” means, but shall not be limited to a PRRS virus strain having a nucleotide sequence substantially identical to SEQ ID NO:1. Preferably a HP PRRS virus is PRRS virus strain having a nucleotide sequence substantially identical to SEQ ID NO:1. Substantial identical to SEQ ID NO:1 shall mean, that the nucleotide sequence of the PRRS virus strain preferably comprises a sequence between 85% and 100% identical to SEQ ID NO:1, preferably under the proviso that the HP PRRS virus is not a PRRS Type II virus as defined herein, for instance that nucleotide homology is less than 91%, preferably, less than 92%, 93%, 94%, 95%, 96%, 97%, 98% or 99% homology in ORF 5 to VR2332 as reference virus isolate. The HP PRRS virus strain nucleotide sequence is preferably greater than 80%, 81%, 82%, 83%, 84%, 85%, 86%, 87%, 88%, or 89% identical to SEQ ID NO:1, likewise preferably under the proviso that the HP PRRS virus is not a PRRS Type II virus as defined herein, for instance that nucleotide homology is less than 91%, preferably, less than 92%, 93%, 94%, 95%, 96%, 97%, 98% or 99% homology in ORF 5 to VR2332 as reference virus isolate. Still more preferably, the PRRS virus strain nucleotide sequence is greater than 90%, 91%, 92%, 93%, 94%, 95%, 96%, 97%, 98% or greater than 99% identical to SEQ ID NO:1 preferably under the proviso that the HP PRRS virus is not a PRRS Type II virus as defined herein, for instance that nucleotide homology is less than 91%, preferably, less than 92%, 93%, 94%, 95%, 96%, 97%, 98% or 99% homology in ORF 5 to VR2332 as reference virus isolate.

The term HP PRRS virus also means any PRRS virus strains, having a defined modification within the NSP2 protein. According to this definition, a HP PRRS virus strain is a PRRS virus strain that encodes a NSP2 protein, wherein the amino acid corresponding to leucine at amino acid position 482 of SEQ ID NO:2 is deleted and which causes the clinical sign of high fever. Alternatively, or in addition to the deleted leucine at amino acid position of SEQ ID NO:2, amino acids corresponding to amino acids 534 to 562 of SEQ ID NO:2 may be deleted from the PRRS virus-encoded NSP2 protein. In this context, SEQ ID NO:2 shall also be understood in a exemplarily manner and the term NSP2 protein shall not be limited to a the NSP2 protein of SEQ ID NO:2. Based on the teaching above, a person skilled in the art can easily identify any corresponding modification in a PRRS virus strains, having a NSP2 protein sequence that is different from the sequence of SEQ ID NO:2, but showing the same modification, which means a deletion of the leucine that corresponds to the leucine at position 482 of SEQ ID NO:2 and/or a deletion of the amino acids which correspond to the amino acids 534 to 562 of SEQ ID NO:2.

Furthermore the term HP PRRS virus may also mean a PRRS virus strain having a nucleotide sequence substantially identical to SEQ ID NO:1 (as defined above) and encoding a NSP2 protein, wherein the amino acid corresponding to leucine at amino acid position 482 of SEQ ID NO:2 and/or the amino acids corresponding to amino acids 534 to 562 of SEQ ID NO:2 are deleted from the PRRS virus-encoded NSP2 protein.

Furthermore the term HP PRRS virus refers to HP PRRS virus that is a PRRS virus strain having a nucleotide sequence substantially identical to SEQ ID NO:1, under the proviso that the HP PRRS virus is not a PRRS Type II virus as defined herein, for instance that nucleotide homology is less than 91%, preferably, less than 92%, 93%, 94%, 95%, 96%, 97%, 98% or 99% homology in ORF 5 to VR2332 as reference virus isolate (as defined above) and encodes a NSP2 protein, wherein the amino acid corresponding to leucine at amino acid position 482 of SEQ ID NO:2 and/or the amino acids corresponding to amino acids 534 to 562 of SEQ ID NO:2 are deleted from the PRRS virus-encoded NSP2 protein.

Furthermore the term HP PRRS virus refers to an HP PRRS virus that is a PRRS virus strain having a nucleotide sequence substantially identical to SEQ ID NO:1, preferably under the proviso that the HP PRRS virus is not a PRRS Type 2 virus as defined herein, for instance that nucleotide homology is less than 91%, preferably, less than 92%, 93%, 94%, 95%, 96%, 97%, 98% or 99% homology in ORF 5 to VR2332 as reference virus isolate (as defined above) and encodes a NSP2 protein, wherein antibodies with reactivity to peptides corresponding to aa positions 536-550 or 546-560 or 476-490 show no reactivity.

Furthermore, the following PRRS virus isolates are known to be HP PRRS virus strains. Consequently the term HP PRRS virus strain, as used herein shall include any of these virus strains as well as any descendant thereof: HP PRRS virus strain AH-1; AHCFSH; AHCFZC; BB07; BD-8; BQ07; CL07; CX07; CZ07; FY060915; FY080108; GC-2; GCH-3; GD1; GD2; GD2007; GD3; GD4; GDSD1; GDY1-2007; GDY2-2007; GDYF1; GS2008; GXHZ12; GXHZ13; GXHZ14; GXHZ16; GXHZ19; GXHZ2; GXHZ21; GXHZ4; GXLZ5; GXLZ7; GY; GZCJ; GZDJ; GZHW1; GZHW2; GZHX; GZJS; GZKB; GZKY; GZLJ1; GZWB; GZWM; GZZB; Hainan-1; Hainan-2; HB1; HB2; HB3; HB-Tsh1; HB-Xt1; HEN46; HeN-KF; HeN-LH; HeN-LY; HLJDF; HLJMZ1; HLJMZ2; HLJMZ3; HLJZY; HM-1; HN2; HN2007; HN3; HNId; HNIy; HNLY01; HNNX01; HNPJ01; HNsp; HNXT1; HNyy; HNyz; HQ-5; HQ-6; HUB; HuN; HUN1; HUN11; HUN15; HUN16; HUN17; HUN2; HUN3; HUN4; HUN5; HUN6; HUN7; Hunan-1; Hunan-2; Hunan-3; HUNH2; HUNH4; HuNhl; HUNL1; HUNX4; HZ061226; HZ070105; Jiangsu-1; Jiangsu-2; Jiangsu-3; Jiangxi-2; Jiangxi-4; JLYS; JN; JX1; JX143; JX2; JX-2; JX2006; JX3; JX4; JX5; JXA1; KS06; LC07; LJ; LS06; LS-4; LY07; NB070319; SC07; SD; SD14; SDWF2; SH02; ST-7; SX2007; SY0608; TJDMJ; TJZHJ2; TJZHJ3; TQ; TQ07; TWO7; WF07; XJ07; XL2008; YN2008; YNBS; YNDL; YNMG; YNWS; YNYS; YNYX1; YNYX3; ZJ06; ZJCJ; ZJWL; ZX07; ZS070921. Descendant means but shall not be limited to a virus isolate that originates from any of the parent viruses listed above and having a nucleotide sequence identity of greater than 86%, 87%, 88%, 89%, 90%, 91%, 92%, 93%, 94%, 95%, 96%, 97%, 98% and 99% with the corresponding parent virus strain.

The term “PRRS Type II virus” means, but shall not be limited to, a PRRS virus strain that is substantially identical to the virus isolate deposited as ATCC-VR2332 or any descendant of the virus isolate deposited as ATCC-VR2332. Substantially identical, as used herein, means that the nucleotide sequence coding for the ORF5 protein is between 85% and 100% identical to the nucleotide sequence of virus isolate deposited as ATCC-VR2332 and as defined in SEQ ID NO:3. The ORF5 nucleotide sequence is preferably greater than 86%, 87%, 88%, or 89% identical to SEQ ID NO:3. Still more preferably, the ORF5 nucleotide sequence is greater than 90%, 91%, 92%, 93%, 94%, 95%, 96%, 97%, 98% or greater than 99% identical to SEQ ID NO:3. Preferably, a PRRS Type II virus as used herein is a PRRS virus strain that is substantially identical to the virus isolate deposited as ATCC-VR2332 or any descendant of the virus isolate deposited as ATCC-VR2332 (as defined above), but does not have a deletion within the NSP2 gene of the amino acids which correspond to the amino acids 534 to 562 of SEQ ID NO:2. The complete sequence of PRRS virus ATCC-VR2332 can be found under Gen Bank accession no. U87392.

The term PRRS Type II virus shall also include any attenuated virus that originated from any of the above-mentioned PRRS Type II virus strains. For instance, the term PRRS Type II virus shall also include attenuated PRRS Type II virus deposited as ATCC-VR2495. Furthermore, an attenuated PRRS Type II virus may be any attenuated descendant of the virus isolate deposited as ATCC-VR2332. In some preferred forms, the PRRS Type II virus and a pharmaceutically acceptable carrier may be Ingelvac® PRRS MLV vaccine (Serial No JA-A64A-149) from Boehringer Ingelheim Vetmedica, Inc. (St. Joseph, Mo.). The term PRRS Type II virus may also include the isolates known as HB-1; BJ-4; CH-1a; CH-1R; CH-1R01; HB-2; HN1; HT06; HZ07; L505; LY03; NH04; PL97-1; S1; SH061130; SX071226; TWO7-1; WF03; XX03; ZJJ04; ZJJ05; ZJJ07, which are non-HP PRRS strains of Chinese Origin.

Another aspect of the present invention provided herein includes a method of prophylaxis of swine of infection with HP PRRS comprising administering to a pig in need thereof an immunogenic composition comprising an effective amount of an PRRS Type II virus, preferably an attenuated PRRS Type II virus, wherein said PRRS type II virus is a PRRS virus strain that is substantially identical to the virus isolate deposited as ATCC-VR2332 or any descendant of the virus isolate deposited as ATCC-VR2332. Preferably, that PRRS type II virus does not have a deletion within the NSP2 gene of the amino acids which correspond to the amino acids 534 to 562 of SEQ ID NO:2. Even more preferred that PRRS type II virus is attenuated PRRS Type II virus deposited as ATCC-VR2495. Furthermore, the PRRS type II virus is that of Ingelvac® PRRS MLV vaccine (Serial No JA-A64A-149).

An effective amount of the PRRS Type II virus may be an amount of the virus that elicits or is able to elicit an immune response in an animal, to which the effective dose of the virus is administered. The amount that is effective may depend on the ingredients of the vaccine and the schedule of administration. If an inactivated virus or a modified live virus preparation is used, an amount of the vaccine containing about 10^{2.0} to about 10^{9.0} TCID₅₀ (tissue culture infective dose 50% end point), more preferably 10^{3.0} to about 10^{4.0} TCID₅₀, and still more preferably from about 10^{4.0} to about 10^{8.0} TCID₅₀ per dose may be recommended.

The herein described PRRS Type II virus may be used as an inactivated whole killed virus or in an attenuated form of a PRRS Type II virus for the prophylaxis of swine of the effects of a high fever disease as described herein. In addition, subunits, including immunogenic fragments or fractions of the PRRS Type II virus, may also be used for the prophylaxis of swine of the effects of a high fever disease.

The herein described attenuated PRRS Type II virus may be a modified live vaccine (MLV) comprising one or more of the strains noted above alive in a pharmaceutically acceptable carrier. In addition, or alternatively, inactivated virus may be used to prepare killed vaccine (KV) as described above. MLV may be formulated to allow administration of between 10¹ to 10⁷ viral particles, more preferably from 10³ to 10⁵ viral particles, and still more preferably from 10⁴ to 10⁵ viral particles per dose. KV may be formulated based on a pre-inactivation titre of between 10³ to 10¹⁰, 10⁴ to 10⁹, 10⁵ to 10⁸, or 10⁶ to 10⁷ viral particles per dose.

The PRRS Type II virus, preferably the attenuated PRRS Type II virus, may be administered to a pig prior to the pig's exposure to a PRRS virus strain that causes HP PRRS as a prophylactic, concomitant with the pig's exposure to a PRRS virus strain that causes HP PRRS, or as a treatment after a target pig is exposed to a PRRS virus strain that causes HP PRRS. The target pig may exhibit one or more clinical signs or common symptoms of HP PRRS or a high fever disease form as described above. A target pig may be particularly susceptible to a high fever disease associated with HP PRRS. A target pig may be particularly susceptible to HP PRRS. The target pig may be susceptible to HP PRRS because of an immunodeficiency. The target pig may be susceptible to HP PRRS because of where the pig is farmed. A susceptible pig may be farmed in China. The susceptible pig may be farmed in a province of China such as the Jiangxi Province, the Hebei Province, or Shanghai City. See Tian et al., PloS ONE. 2007; 2(6):e526, the contents of which are incorporated herein by reference. The attenuated PRRS Type II virus may be administered via injection, via inhalation, or via an implant, with injection being particularly preferred. Depending on the desired duration and effectiveness of the vaccination or treatment, the PRRS Type II virus, preferably the attenuated PRRS Type II virus, may be administered once or several times, also intermittently, for example on a daily basis for several days, weeks or months and in different dosages. Of these, a single dose administration is preferred. Injection may be peripherally or at a central vein at a desired amount, or alternatively, continuously infused. The PRRS Type II virus, preferably the attenuated PRRS Type II virus, may be administered orally, parenterally, subcutaneously, intramuscularly, intradermally, sublingually, transdermally, rectally, transmucosally, topically via inhalation, via buccal administration, or combinations thereof. The PRRS Type II virus, preferably the attenuated PRRS Type II virus, may also be administered in the form of an implant, which may allow slow release of the attenuated virus. For intramuscular injection, a volume of between 0.5 mL and 3 mL, more preferably between 1 mL and 2.5 mL, still more preferably between 1.5 ml and 2 mL may be applied. An intramuscular injection of 2 mL is most preferred. For intradermal injection, a volume of between 0.05 mL and 1 mL, more preferably between 0.1 mL and 0.8 mL, still more preferably between 0.1 and 0.5 mL, even more preferably between 0.2 and 0.4 mL is administered. Most preferably, an intradermal injection of 0.2 mL may be applied. PRRS Type II virus volumes of between 0.5 mL and 5 mL, more preferably between 1 mL and 4 mL, still more preferably between 2 mL and 3 mL may be intranasally applied. Most preferably, a volume of 3 mL may be intranasally applied.

The pharmaceutically acceptable carrier may include any and all solvents, dispersion media, coatings, stabilizing agents, diluents, preservatives, antibacterial and antifungal agents, isotonic agents, adsorption delaying agents, and the like.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). JohnWiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Carbopol 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, α-tocopherol acetate, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Also provided herein is a method of producing an attenuated PRRS Type II virus that is capable of treating or immunizing a target pig against HP PRRS. The method may comprise one or more of the following steps: (a) passaging ATCC-VR2332 or any PRRS Type II substantially identical to ATCC-VR2332, as described below, to modify and render the virus avirulent and capable of immunizing the target pig against HP PRRS, (b) harvesting the production virus cells or cell culture, (c) adding a stabilizing agent to the production virus culture; and/or (d) lyophilizing the production virus culture. Virus passage may encompass classical propagation and selection techniques; for example, continued propagation in suitable host cells to extend the attenuated phenotype. Passaging, may result in a viral strain that has acquired mutations, many of which will not alter properties of the parent strain significantly. The attenuated PRRS Type II virus may be the result of ATCC-VR2332 or any PRRS Type II substantially identical to ATCC-VR2332 having been passaged at least 60, 65, 70, 75, 80, or more times in a host cell. The attenuated PRRS Type II virus may be the result of ATCC-VR2332 or any PRRS Type II substantially identical to ATCC-VR2332 having been passaged between 50 and 100 times, between 60 and 90 times, between 70 and 80 times, or between 65 and 75 times in a host cell. The attenuated PRRS Type II virus may be the result of ATCC-VR2332 or any PRRS Type II substantially identical to ATCC-VR2332 having been passaged 70 or 75 times in a host cell. A suitable host cell may include a simian cell line, Vero cells, or porcine alveolar macrophages. A preferred simian cell line is MA-104. The host cell may be a cell culture. The cell line may be infected with the virus to be passaged. Each passage may require incubating the resultant virus infected cell line or cell culture at a temperature between 34° C. and 40° C., more preferably between 35° C. and 39° C., still more preferably between 36° C. and 38° C., and even more preferably between 35° C. and 37° C. Most preferably, each passage may require incubating the resultant virus infected cell line or cell culture at a temperature of 37° C. The step of harvesting may include freezing the virus-infected cell culture. Lyophilizing may include subliming moisture from a frozen sample of the virus-infected cell culture.

Virus modification may also be used to produce an attenuated PRRS Type II virus and may be achieved by directed mutation of the nucleic acid sequence of the virus strain by suitable genetic engineering techniques. Such techniques may employ construction of a full-length complementary nucleic acid copy of the viral genome that may be modified by nucleic acid recombination and manipulation methods. Such methods may employ site directed mutagenesis. Antigenic sites or enzymatic properties of viral proteins then therefore be modified.

Also provided herein is a kit for performing any of the foregoing described methods. The kit may comprise a container, an immunogenic composition preferably comprising attenuated PRRS Type II virus, a pharmaceutically acceptable carrier, an adjuvant, and instructions for administering the immunogenic composition to an animal in need thereof in order to lessen the incidence of or severity of clinical signs or effects of PRRS infection, and preferably high fever disease forms of PRRS or HP-PRRS. The kit may further comprise a means for injection and/or a means for another form of administration. The kit may still further comprise a solvent. The attenuated vaccine may be freeze dried and may be reconstituted with the solvent, resulting in a solution for injection and/or inhalation. The solvent may be water, physiological saline, buffer, or an adjuvanting solvent. The kit may comprise separate containers for containing the attenuated virus, solvent, and/or pharmaceutically acceptable carrier. The instructions may be a leaflet and/or a label affixed to one or more of the containers.

### BRIEF DESCRIPTION OF THE DRAWINGS

FIG. 1 is a depiction of how lungs were scored and evaluated for percentage of area affected by visible pneumonia;

FIG. 2 is a graph comparing the rectal temperature of pigs in vaccinated and non-vaccinated groups;

FIG. 3 is a graph illustrating a comparison of the mean S/P ratio of pigs in vaccinated and non-vaccinated groups wherein the mean group ELISA S/P ratio was used to measure a respective group's serological response to PRRSV;

FIG. 4 is a graph illustrating a comparison of group average clinical scores of pigs in vaccinated and non-vaccinated groups wherein respiratory disease scores from vaccinated and non-vaccinated groups of pigs were recorded;

FIG. 5 is a graph comparing the average daily weight gain (ADG) of pigs in vaccinated and non-vaccinated groups; and,

FIG. 6; is a graph illustrating a summary of the percentage of PRRSV RT-PCR positive sera in MLV-vaccinated pigs and non-vaccinated/challenged pigs.

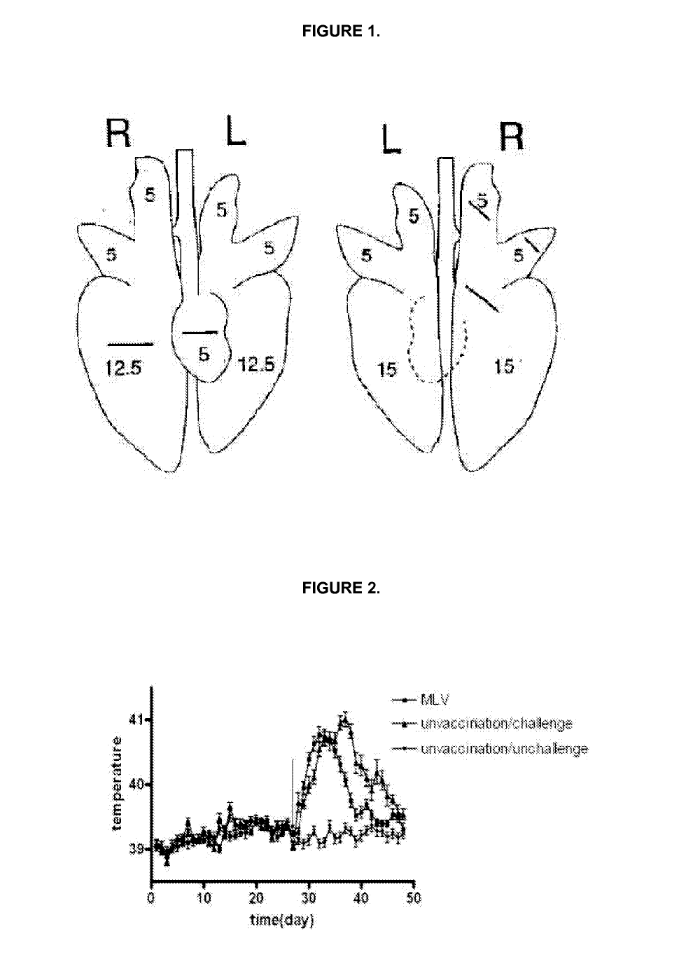

### DETAILED DESCRIPTION OF THE INVENTION

### Definitions

The terminology used herein is for the purpose of describing particular embodiments only and is not intended to be limiting. As used in the specification and the appended claims, the singular forms “a,” “and” and “the” include plural references unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Attenuated virus” as used herein may mean an avirulent virus that does not cause clinical signs of PRRS disease but is capable of inducing an immune response in the target mammal, but may also mean that the clinical signs are reduced in incidence or severity in animals infected with the attenuated virus in comparison with a “control group” of animals infected with non-attenuated PRRS virus and not receiving the attenuated virus. In this context, the term “reduce/reduced” means a reduction of at least 10%, preferably 25%, even more preferably 50%, most preferably of more than 100% as compared to the control group as defined above.

“Immunogenic fragment” as used herein may mean a portion of peptide or polypeptide or nucleic acid sequence of PRRS Type II virus that can elicit an immune response in the host including a cellular and/or antibody-mediated immune response to PRRSV.

“Identical” or “identity” as used herein in the context of two or more polypeptide or nucleotide sequences, may mean that the sequences have a specified percentage of residues or nucleotides that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

PRRS isolate ATCC VR-2332 was deposited with the American Type Culture Collection in Rockville, Md., in accordance with the Budapest Treaty on Jul. 7, 1992, and given the accession No. ATCC VR-2332.

PRRS isolate VR-2495 was deposited with the American Type Culture Collection in Rockville, Md., in accordance with the Budapest Treaty on Jan. 28, 1995, and given the accession No. ATCC VR-2495.

“Immunogenic composition” or “vaccine” as used herein, mean a composition comprising PRRS Type II virus (MLV or killed virus) or any immunogenic fragment or fraction thereof, preferably attenuated PRRS Type II virus, such as Ingelvac PRRS MLV or Ingelvac PRRS ATP, which elicits an “immunological response” in the host of a cellular and/or antibody-mediated immune response to PRRSV. Preferably, this immunogenic composition is capable of conferring protective immunity against PRRSV infection and the clinical signs associated therewith.

“To elicit an immunological response or immune response” as used herein means any cellular and/or antibody-mediated immune response to an immunogenic composition or vaccine administered to an animal receiving the immunogenic composition or vaccine. Usually, an “immune response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced in comparison to controls that do not receive an administration of the immunogenic composition or vaccine. Such protection will be demonstrated by either a reduction in the incidence of or severity of up to and including a lack of the symptoms associated with host infections as described above.

“Protective immunity” as used herein, means that the resistance in a group of animals to an infection with PRRS, preferably HP PRRS will be enhanced in comparison with a control group of animals infected with HP PRRS but not receiving a PRRS, preferably a PRRS type II containing immunogenic composition or vaccine. The term “enhanced resistance” as used herein means that less than 10%, preferably less than 20%, even more preferably less than 30%, even more preferably less than 40%, even more preferably less than 50%, even more preferably less than 75%, even more preferably less than 100% of the animals receiving the immunogenic composition or vaccine of the invention develop one or more clinical symptoms associated with high fever, preferably caused by HP PRRS as described herein, as compared with a group of animals infected with PRRS but not receiving the immunogenic composition or vaccine.

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Swine,” “pig,” and “piglet” as used herein may be used interchangeably.

“Vaccinate” refers to the administration of the immunogenic composition or vaccine described herein prior to exposure to high fever disease forms of PRRS or HP-PRRS.

“Protect” or “protection” refer to the reduction in severity of or incidence of clinical signs of HP-PRRS infection or high fever disease forms of PRRS as a result of receiving an administration of the immunogenic composition of the present invention. The reduction in severity of or incidence of is in comparison to an animal or group of animals not receiving the immunogenic composition of the present invention.

### Preferred Embodiments

The following examples set forth preferred materials and procedures in accordance with the present invention. Although any materials and methods similar or equivalent to those described herein can be used in the practice or testing of the present invention, the preferred methods, devices, and materials are now described. It is to be understood, however, that these examples are provided by way of illustration only, and nothing therein should be deemed a limitation upon the overall scope of the invention.

### Examples

The below-identified Examples illustrate the highly virulent nature of PRRSV isolate JX143. Ingelvac® PRRS MLV-vaccinated pigs have 100% survival and significantly higher antibody response, lower ratio of clinical PRRS and viremia, less severe lung lesion, fewer, lighter and shorter clinical signs, and a shorter period of high rectal temperature as compared to non-vaccinated pigs after challenge with a highly virulent PRRSV strain.

1. Materials and Methods

1.1 Vaccines and Virus

Ingelvac® PRRS MLV vaccine (Serial No JA-A64A-149) was from Boehringer Ingelheim Vetmedica. Highly virulent PRRSV isolate JX143 was isolated by Shanghai Veterinary Research Institute. PRRSV JX143 tissue culture (105.2TCID50/ml) was diluted five fold with DMEM for pig inoculation.

1.2 Primers and Reagents

Reverse transcription polymerase and DNA ladder were purchased from Tiangen biotechnology company. 2×PCR Mix was from Dongsheng company. Trizol® and primers were from Invitrogen company.

TABLE 1
Primers used for RT-PCR amplification

| Primer name | Sequence |
|---|---|
| SF14413 | 5′- CTGATCGACCTCAAAAGAGTTGTGCTTG -3′ |
| | (SEQ ID NO: 4) |
| SR15497 | 5′- CAATTAAATCTTACCCCCACACGGTCG -3′ |
| | (SEQ ID NO: 5) |
| Qst | 5′-gagtgacgaggactcgagcgcattaaTTTTTTTTTTTTTT -3′ |
| | (SEQ ID NO: 6) |

1.3 Animal Source and Grouping

Fifty (50) pigs 29 days of age were purchased for the trial from Henan Muyuan breeding pig farm. They were confirmed negative for PRRSV and PCV2 by RT-PCR (for PRRSV & PCV 2) and ELISA (an anti-PRRSV kit, IDEXX Laboratory, Inc.) by running each of the three tests on serum samples collected on arrival. The pigs were weighed and randomly assigned to groups 1, 2, or 3, each containing 22, 14 and 14 pigs, respectively. Pigs were then housed in separate rooms according to their group.

1.4 Vaccination and Virus Challenge

The 22 pigs in group 1 (V/C) were vaccinated on day 0 with a single 2 mL dose of Ingelvac® PRRS MLV vaccine intramuscularly. The 14 pigs in group 2 (non-V/C) were injected with 2 mL PBS on day 0. Challenge of group 1 and group 2 pigs occurred on day 28 with the intranasal administration of 3 mL diluted PRRSV JX143. The pigs in group 3 (non-V/non-C) were not vaccinated and not challenged as strict negative control and they were injected with 3 mL DMEM on day 28. Two pigs per group were necroscopied on day 14 and 42 respectively for observation. The remainder of the pigs were necroscopied on day 49.

1.5 Rectal Temperature

Rectal temperature was recorded at the same time everyday from day 0 to day 49 (21 days post challenge).

1.6 Serology

Sera were collected on days 0, 7, 14, 21, 28, 32, 42, and 49 from all pigs and tested for anti-PRRSV antibody using an IDEXX PRRSV ELISA kit.

1.7 Clinical Evaluation

The pigs were monitored daily from day 0 to day 49 and scored for severity of behavioural changes and clinical respiratory signs including respiration and cough. The scoring system of clinical signs is shown in Table 2.

TABLE 2
Scoring system of clinical signs.

| Severity of | Score aspect | | |
|---|---|---|---|
| clinical signs | Respiration* | Behavior* | Cough* |
| Normal | 1 | 1 | 1 |
| Slight | 2 | 2 | 2 |
| Serious | 3 | 3 | 3 |
| Death | 4 | 4 | 4 |

*Respiration: Score 2 (Slight) corresponds to superficial respiration, nasal discharge, abdominal “thumping” respiration when stimulated. Score 3 (Serious) corresponds to rapid and superficial respiration, nasal discharge, open mouth breathing, abdominal “thumping” respiration.
*Behavior: 1. skin of mouth, nose, ears and inside of legs turn red, congestion, red spot, papula 2. depressing, rough hair coat. 3. anorexia 4. lameness, tremor, convulsion 5. emaciated. Score 2 corresponds to one or two items of symptom as described above. Score 3 corresponds to three items or above symptom as described above..
*Cough: Score 2 corresponds to non-productive cough. Score 3 corresponds to productive cough.

1.8 Evaluation of productivity

Weight of all the pigs were recorded on day 0 (before vaccination), day 28 (before challenge), day 49 (21 day post challenge).

1.9 Efficacy of Ingelvac® PRRS MLV was assessed by evaluating the clinical signs, lung lesion scores and rectal temperatures following challenge in vaccinated pigs as compared to the challenge control and negative control groups. Pigs were considered to be clinically-affected by PRRS when 1) high rectal temperature (41° C.) for more than 3 days, 2) depression, anorexia, conjunctivitis, cough, respiratory disease, and 3) pneumonia were evident.

1.10 Lung Lesions

Necropsy was performed at day 49 (21 days after PRRSV challenge). For each pig, the lungs were evaluated for percentage of area (0 to 100%) affected by grossly visible pneumonia (edema, congestion, hemorrhage, meaty and firm fibrous structure) in a blind fashion.

1.11 PRRSV RNA Detection and Quantification

Sera were collected from pigs in group 1 (V/C) on days 0, 7, 14, 21, 28, 32, 35, 42 and from pigs in group 2 (non-V/C) on days 28, 32, 35 and 42. RNA extraction from 140 μL of individual serum samples was performed using a QIAamp viral RNA mini kit (QIAGEN). RT-PCR was then performed using prime-probe (Invitrogen) combinations (Table 1) specific for the conserved region of PRRSV RNA (Genbank).

Each RT reaction consisted of 12.5 μL of the RNA template, 4 μL of dNTP, 2 μL of 10× Buffer, 0.5 μL of primer Qst and 1 μL of Quant reverse transcriptase. The mixture was incubated in 37° C. water bath for 1 hour and stored at −20° C. PCR was then performed using 1 μL of the RT reaction, 1 μL of each of SF14413 and SR1549 primers, 2 μL of 10× Buffer, 2 μL of dNTP, 5 unit of rTaq polymerase and water to a total volume of 20 μL. The reaction plate was run in a sequence detection system under specific conditions (94° C. for 5 min; then 40 cycles of 94° C. 30 sec, 65° C. 30 sec, 72° C. 75 sec, 40 cycles; finally 72° C. for 10 min). The PCR amplified fragment was separated by agarose gel and detected under ultraviolet light.

1.12 Immunohistochemistry for Detection of PRRSV Antigen.

Immunohistochemistry for detection of PRRSV-specific antigen was performed on formalin-fixed and paraffin-embedded lung tissue sections made within 48 hours after necropsy by using PRRSV anti-N-protein monoclonal antibody (SR30 or SDOW17) and secondary conjugated antibody.

2. Results

2.1 Rectal Temperature Change Post Challenge.

Following inoculation with the highly virulent PRRSV JX143, rectal temperature in pigs of both the vaccinated group and the non-vaccinated group quickly increased. The peak temperature was 41° C. and 75% of the pigs were febrile immediately following inoculation of the challenge virus. Rectal temperatures declined to pre-challenge levels within 10 days in the vaccinated pigs while pigs in the non-V/C group had a longer febrile period and increased rectal temperatures were present significantly longer (FIG. 2).

2.2 Serological Response.

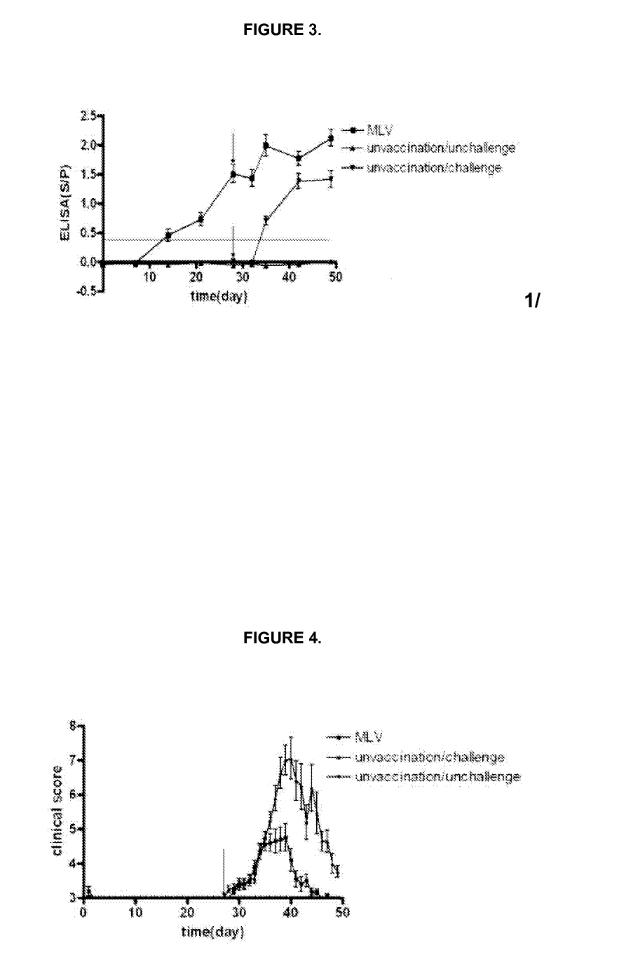

The mean group ELISA S/P reaction was used to measure a respective group's serological response to PRRSV (FIG. 3). The negative-control pigs remained negative for PRRSV antibodies throughout the study. In the V/C group, the antibody was firstly detected at 10-14 days post-vaccination, and S/P ratio 0.4 occurred at 14 days post inoculation (p.i.) with 8 of 20 pigs positive, and at 21 days p.i., 13 of 20 pigs were positive in the V/C group. The highest S/P ratio in the V/C group was observed after challenge and remained high to the end of the study (ELISA S/P=2). The non-V/C pigs seroconverted quickly following challenge; at 7 days p.i. 9 of the 12 pigs were positive with S/P ratio 0.4.

2.3 Clinical Signs.

The respiratory disease scores from the V/C group and the non-V/C group were recorded (FIG. 4). Following challenge, 5 of the 20 pigs in the V/C group exhibited respiratory signs and cough and 2 pigs exhibited abdominal “thumping” respiration. Eight of the 12 pigs in the non-V/C group died before 21 days p.i., and the remaining pigs in the non-V/C group showed serious respiratory signs and cough with abdominal “thumping” respiration. The non-V/C pigs scored above 6 consecutively for 10 days and the highest score reached 7. The V/C pigs did not present significant clinical signs and their high average score was in the 4-5 range for 7 consecutive days. As strict negative controls, the non-V/non-C pigs showed no clinical signs and their average score was 3 (normal).

2.4 Average Daily Weight Gain Pre- and Post-Challenge

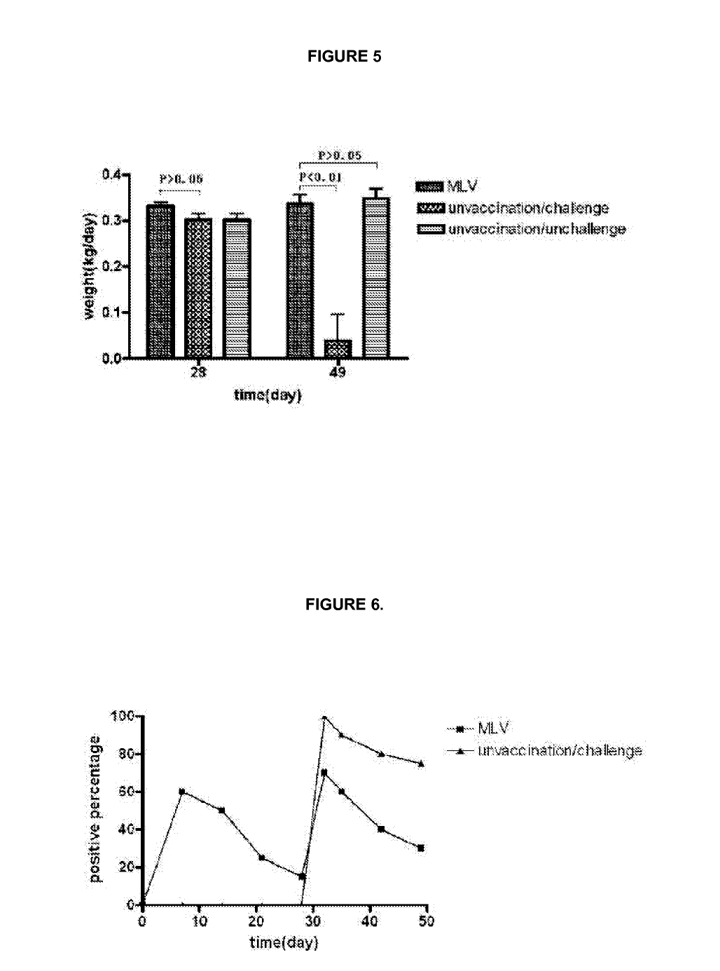

The average daily weight gain (ADG) is summarized in FIG. 5. From day 0 to 28, the ADG was not significantly different between the vaccinated pigs (0.3301±0.0414 Kg) and non-vaccinated pigs (0.3008±0.0653 Kg). From day 28 (before day of challenge) to day 49, the vaccinated pigs had a similar ADG as the non-V/non-C control pigs (0.3373±0.0800 kg vs. 0.3484±0.0890 kg) while the non-V/C pigs had sharply a much lower ADG (0.0392±0.2398).

2.5 Efficacy of Vaccination.

Following challenge, the MLV-vaccinated pigs displayed clinical signs shortly after challenge with an average clinical-sign score of 5, and more than 3 pigs had high rectal temperature (41° C.) and lung lesions. As defined by the criteria mentioned in the methods section, 25% (5/20) of the MLV-vaccinated pigs had PRRS and 75% (15/20) of the pigs were protected. In contrast, all of the non-vaccinated pigs had PRRS after challenge and 8 pigs died before necropsy.

TABLE 3
Efficacy of MLV vaccination
Group

| | Pigs | | | Challenge |
|---|---|---|---|---|
| | challenged | Pigs died | Sick pigs | result |
| MLV-V | 20 | 0 | 5 (25%) | 100% |
| | | | | survival |
| Non- | 12 | 8 | 12 (100%) | 33% survival |
| v/challenge | | | | |

2.6 Gross Lesions.

At necropsy, the four surviving pigs in the non-V/C group presented gross lesions including lung failure/collapse, mottled tan, congestion, lymph of groin, jowl, mesentery edema and congestion, and liver necrosis in some. A few pigs in the V/C group had similar but less severe lesions. The non-V/non-C control pigs did not have gross lesions.

2.7 Gross Lung Lesion Scores

The gross lung lesion score of MLV-vaccinated pigs was significantly lower than that of the non-V/C pigs, suggesting MLV provided good protection against high pathogenic PRRSV inoculation (Table 4, wherein the range for the macroscopic lung lesion incidence is 0 to 100%).

TABLE 4
Comparison of severity of macroscopic lung lesion in pigs of different
groups

| Day post | Group | | |
|---|---|---|---|
| vaccination | MLV-V/C | Non-V/C | Non-V/non-C |
| 14 | 0.500 ± 2.00 | 0.30 ± 2.30 | |
| 42 | 28.58 ± 16.15 | 75.25 ± 7.27 | 0.25 ± 1.00 |
| 49 | 19.12 ± 8.37 | 69.6 ± 12.97 | 0.30 ± 0.50 |

2.8 Detection of Viremia

The percentage of PRRSV RT-PCR positive sera is summarized in FIG. 6. Viremia was detected in 60% of MLV-vaccinated pigs 7 days post vaccination, which declined to 20% before challenge. Post challenge, 70% of the vaccinated pigs had viremia, which declined to 60% at 7 days p.i. and 20% were viremic at 21 days p.i. In contrast, 100% of the non-V/C pigs had viremia following challenge, viremia remained high following challenge, and 70% of the non-V/C pigs were viremic at 21 days post-challenge.

2.9 Antigen Detection by Immunohistochemistry.

Microscopic lung lesion was observed under a microscope. PRRSV-infected cells were seen in all pigs challenged. The MLV-V/C pigs had fewer PRRSV-infected cells. The numbers of total cells and PRRSV-infected cells were recorded in different areas. The cell infection ratio differed significantly between the groups: 23.34±4.691 for the non-V/C pigs, 9.36±8.069 for the V/C pigs and 0.24±0.114 for the non-V/non-C pigs (Table 5).

TABLE 5
PRRSV-infected cells by immunohistochemistry of paraffin-embedded
blocks pig lung.

| Ratio of PRRSV- | Groups | | |
|---|---|---|---|
| infected cell | MLV-V | Non-v/challenge | Non-v/non-c |
| % | 9.36 ± 8.069 | 23.34 ± 4.691 | 0.24 ± 0.114 |

Sequences

| SEQ ID NO: 1 | |
|---|---|
| (SEQUENCE OF JX143, GENBANK EU708726) | |
| 1 | atgacgtata ggtgttggct ctatgccacg gcatttgtat tgtcaggagc tgtgaccatt |
| 61 | ggcacagccc aaaacttgct gcacgggaac accctcctgt gacagccctc ttcaggggga |
| 121 | ttaggggtct gtccctaaca ccttgcttcc ggagttgcac tgttttacgg tctctccacc |
| 181 | cctttaacca tgtctgggat acttgatcgg tgcacgtgta cccccaatgc tagggtgttt |
| 241 | gtggcggagg gccaggtcta ctgcacacga tgtctcagtg cacggtctct ccttcctctg |
| 301 | aatctccaag ttcctgagct tggggtgctg ggtctatttt ataggcccga agagccactc |
| 361 | cggtggacgt tgccacgtgc attccccact gtcgagtgct cccccgccgg ggcctgttgg |
| 421 | ctttctgcga tttttccgat tgcacgaatg actagtggaa acctgaactt tcaacaaaga |
| 481 | atggtgcggg tcgcagctga aatctacaga cccggccaac tcacccctac agttctaaag |
| 541 | actctacaag tttatgaacg gggttgtcgc tggtacccca ttgtcgggcc cgtccctggg |
| 601 | gtgggcgttt acgccaactc cctgcatgtg agtgacaaac ctttcccggg agcaactcat |
| 661 | gtgttaacca acttgccgct cccgcagagg cccaaacctg aggacttttg cccttttgag |
| 721 | tgtgctatgg ctgacgtcta tgacattggt cgtggcgctg tcatgtatgt ggccggagga |
| 781 | aaggtctctt gggcccctcg tggtgggaat gaagtgaaat ttgaacctgt tcccaaggag |
| 841 | ttgaagttgg ttgcgaaccg actccacacc tccttcccgc cccatcacgt agtggacatg |
| 901 | tccgagttta ccttcatgac ccctgggagt ggtgtctcca tgcgggttga gtaccaatac |
| 961 | ggctgcctcc ctgctgacac tgtccctgaa ggaaactgct ggtggcgctt gtttgactcg |
| 1021 | ctcccaccgg aagttcagta caaagaaatt cgccatgcta accaatttgg ctatcaaacc |
| 1081 | aagcatggtg tccctggcaa gtacctacag cggaggctgc aagttaatgg tcttcgggca |
| 1141 | gtgaccgaca cacatggacc tatcgtcata cagtacttct ctgttaagga gagttggatc |
| 1201 | cgccacctga agttggtgga agaacccagc ctccccgggt ttgaggatct cctcagaatc |
| 1261 | agggttgagc ccaatacgtc accactggct agaaaggatg agaagatttt ccggtttggc |
| 1321 | agtcataagt ggtacggtgc cggaaagaga gcaaggaaaa cacgctctgg tgcgactact |
| 1381 | atggtcgctc atcacgcttc gtccgctcat gaaacccggc aggccacgaa gcacgagggt |
| 1441 | gccggcgcta acaaggccga gcatctcaag cgctactctc cgcctgccga agggaactgt |
| 1501 | ggttggcact gcatttccgc catcgccaac cggatggtga attccaactt tgagaccacc |
| 1561 | cttcctgaaa gggtaaggcc ttcagatgac tgggccactg acgaggatct tgtgaacacc |
| 1621 | atccaaatcc tcaggctccc tgcggccttg gacaggaacg gcgcttgcgg tagcgccaag |
| 1681 | tacgtgctta aactggaggg tgagcattgg actgtctctg tgatccctgg gatgtcccct |
| 1741 | actttgctcc cccttgaatg tgttcagggt tgttgtgagc ataagggcgg tcttgtttcc |
| 1801 | ccggatgcgg tcgaaatttc cggatttgat cctgcctgcc ttgaccgact ggctaaggta |
| 1861 | atgcacttgc ctagcagtac catcccagcc gctctggccg aattgtccga cgactccaac |
| 1921 | cgtccggttt ccccggccgc tactacgtgg actgtttcgc aattctatgc tcgtcataga |
| 1981 | ggaggagatc atcatgacca ggtgtgctta gggaaaatca tcagcctttg tcaagttatt |
| 2041 | gaggattgct gctgccatca gaataaaacc aaccgggcta ctccggaaga ggtcgcggca |
| 2101 | aagattgatc agtacctccg tggcgcaaca agtcttgagg aatgcttggc caaacttgag |
| 2161 | agagtttccc cgccgagcgc tgcggacacc tcctttgatt ggaatgttgt gcttcctggg |
| 2221 | gttgaggcgg cgaatcagac aaccgaacaa cctcacgtca actcatgctg caccctggtc |
| 2281 | cctcccgtga ctcaagagcc tttgggcaag gactcggtcc ctctgaccgc cttctcactg |
| 2341 | tccaattgct attaccctgc acaaggtgac gaggttcatc accgtgagag gttaaattcc |
| 2401 | gtactctcta agttggaaga ggttgtcctg gaagaatatg ggctcatgtc cactggactt |
| 2461 | ggcccgcgac ccgtgctgcc gagcgggctc gacgagctta aagaccagat ggaggaggat |
| 2521 | ctgctaaaac tagccaacac ccaggcgact tcagaaatga tggcctgggc agctgagcag |
| 2581 | gtcgatttaa aagcttgggt caaaagctac ccgcggtgga cacctccacc ccctccacca |
| 2641 | agagttcaac ctcgcagaac aaagtctgtc aaaagcttgc cagaggacaa gcctgtccct |
| 2701 | gctccgcgca ggaaggtcag atccgattgc ggcagcccgg ttttgatggg cgacaatgtc |
| 2761 | cctaacggtt cggaagaaac tgtcggtggt cccctcaatt ttccgacacc atccgagccg |
| 2821 | atgacaccta tgagtgagcc cgtacttgtg cccgcgtcgc gacgtgtccc caagctgatg |
| 2881 | acacctttga gtgggtcggc accagttcct gcaccgcgta gaactgtgac aacaacgctg |
| 2941 | acgcaccagg atgagcctct ggatttgtct gcgtcctcac agacggaata tgaggctttc |
| 3001 | cccctagcac cgtcgcagaa catgggcatc ctggaggcgg gggggcaaga agctgaggaa |
| 3061 | gtcctgagtg aaatctcgga tatactaaat gacaccaacc ctgcacctgt gtcatcaagc |
| 3121 | agctccctgt caagtgttaa gatcacacgc ccaaaatact cagctcaagc catcatcgac |
| 3181 | tctggcgggc tttgcagtgg gcatctccaa aaggaaaaag aagcatgcct cagcatcatg |
| 3241 | cgtgaggctt gtgatgcgtc caagcttagt gatcctgcta cgcaggagtg gctctctcgc |
| 3301 | atgtgggata gggttgacat gctgacttgg cgcaacacgt ctgcttacca ggcgtttcgc |
| 3361 | atcttaaatg gcaggtttga gtttctccca aagatgattc tcgagacacc gccgccccac |
| 3421 | ccgtgcgggt ttgtgatgtt acctcacacg cctgcacctt ccgtgagtgc agagagtgat |
| 3481 | ctcaccattg gttcagtggc caccgaggat gttccacgca tcctcgggaa aataggagac |
| 3541 | actgacgagc tgcttgaccg gggtccctcg gcaccctcca agggagaacc ggtctgtgac |
| 3601 | caacctgcca aagatccccg gatgtcgccg cgggagtctg acgagagcat aatagctccg |
| 3661 | cccgcagata caggtggtgt cggctcattc actgatttgc cgtcttcaga tggtgtggat |
| 3721 | gtggacgggg gggggccgtt aagaacggta aaaacaaaag caggaaggct cttagaccaa |
| 3781 | ctgagctgcc aggtttttag cctcgtttcc catctcccta ttttcttctc acacctcttc |
| 3841 | aaatctgaca gtggttattc tccgggtgat tggggttttg cagcttttac tctattttgc |
| 3901 | ctctttctat gttacagtta cccattcttc ggttttgctc ccctcttggg tgtattttct |
| 3961 | gggtcttctc ggcgtgtgcg aatgggggtt tttggctgct ggttggcttt tgctgttggt |
| 4021 | ctgttcaagc ctgtgtccga cccagtcggc actgcttgtg agtttgactc gccagagtgt |
| 4081 | aggaacgtcc ttcattcttt tgagcttctc aaaccttggg accctgtccg cagccttgtt |
| 4141 | gtgggccccg tcggtctcgg ccttgccatt cttggcaggt tattgggcgg ggcacgctac |
| 4201 | atctggcact ttttgcttag gcttggcatt gttgcagact gtatcttggc tggagcttat |
| 4261 | gtgctttctc aaggtaggtg taaaaagtgc tggggatctt gtgtaagaac tgctcctaat |
| 4321 | gagatcgcct tcaacgtgtt cccttttaca cgtgcgacca ggtcgtcact catcgacctg |
| 4381 | tgcgatcggt tttgcgcacc aaaaggcatg gaccccattt ttctcgccac tgggtggcgt |
| 4441 | gggtgctgga ccggccggag tcccattgag caaccttctg aaaaacccat cgcgttcgcc |
| 4501 | cagctggatg agaagaggat tacggctaga actgtggtcg ctcagcctta tgatcccaac |
| 4561 | caggccgtaa agtgcttgcg ggtattacag gcgggtgggg cgatggtggc cgaggcagtc |
| 4621 | ccaaaagtgg tcaaagtttc cgctattcca ttccgagctc ctttctttcc cgctggagtg |
| 4681 | aaagttgatc ctgagtgcag aatcgtggtt gatcccgata cttttactac agccctccgg |
| 4741 | tctggctatt ccaccgcgaa cctcgtcctt ggtacggggg actttgccca gctgaatgga |
| 4801 | ctaaagatca ggcaaatttc caagccttca gggggaggcc cacacctcat tgctgccttg |
| 4861 | catgttgcct gctcgatggc gttacacatg cttgctggtg tttatgtaac tgcagtgggg |
| 4921 | tcctgcggta ccggtaccaa cgatccgtgg tgcactaacc cgtttgccgt ccctggctac |
| 4981 | ggacctggct ctctttgcac gtctagattg tgcatctccc aacacggcct caccttgccc |
| 5041 | ttgacagcac ttgtggcggg attcggcctt caagagattg ccttggtcgt tttgattttt |
| 5101 | gtctccatcg gaggcatggc tcataggttg agttgtaagg ctgacatgtt gtgcatctta |
| 5161 | ctcgcaatcg ctagttatgt ttgggtacct cttacctggt tgctttgtgt gtttccttgt |
| 5221 | tggttgcgct gtttctcttt gcaccccctc accatcctat ggttggtgtt tttcttgatt |
| 5281 | tctgtaaata taccctcagg agtcttggcc gtggtgttgt tgatttctct ctggctttta |
| 5341 | ggtcgttata ctaacgttgc tggtcttgtc actccctatg acattcatca ttacaccagt |
| 5401 | ggcccccgcg gtgttgccgc cttggctacc gcaccagatg ggacctactt ggccgctgtc |
| 5461 | cgccgcgctg cgctgactgg tcgtaccatg ctgttcaccc cgtctcagct cgggtccctc |
| 5521 | cttgagggcg ctttcagaac tcaaaagccc tcactgaaca ccgtcaatgt ggtcgggtcc |
| 5581 | tccatgggct ctggcggagt gttcactatt gacgggaaaa tcaagtgcgt gactgccgca |
| 5641 | catgtcctta cgggtaactc agctagggtt tccggggtcg gcttcaatca aatgcttgac |
| 5701 | tttgatgtaa aaggggactt cgccatagct gattgcccga attggcaagg ggttgctccc |
| 5761 | aaggcccagt tctgcgagga tgggtggact ggtcgcgcct attggctgac atcctctggc |
| 5821 | gttgaacccg gtgttattgg gaatgggttc gccttctgct tcaccgcgtg tggcgattct |
| 5881 | ggatccccag tgattaccga agccggtgag cttgtcggcg ttcacacagg atcaaacaaa |
| 5941 | caaggaggag gcattgtcac gcgcccctca ggccagtttt gtaatgtgaa gcccatcaag |
| 6001 | ctgagcgagt tgagtgaatt cttcgctgga cctaaggtcc cgctcggtga tgtgaaaatt |
| 6061 | ggcagtcaca taattaaaga cacatgcgag gtgccttcag atctttgtgc cctgcttgct |
| 6121 | gccaaacccg aactggaagg aggcctttcc acagttcaac ttctgtgtgt gtttttcctc |
| 6181 | ctgtggagaa tgatggggca tgcttggacg cccttggttg ctgtggggtt tttcatcctg |
| 6241 | aatgagattc tcccagctgt cctggtccgg agtgttttct cctttgggat gtttgtgcta |
| 6301 | tcttggctca caccatggtc tgcgcaagtc ctgatgatca ggcttctgac agcagccctt |
| 6361 | aacagaaaca gatggtctct tggtttttac agccttggtg cagtaaccag ttttgtcgca |
| 6421 | gatcttgcgg taactcaagg gcatccgtta caggtggtaa tgaacttaag cacctatgcc |
| 6481 | ttcctgcccc ggatgatggt tgtgacctcg ccagtcccag tgatcgcgtg tggtgttgtg |
| 6541 | cacctccttg ccataatttt gtacttgttt aagtaccgct gccttcacaa tgtccttgtt |
| 6601 | ggcgatgggg tgttctcttc ggctttcttc ttgcgatact ttgccgaggg aaagttgagg |
| 6661 | gaaggggtgt cgcaatcctg cgggatgagt catgagtcgc tgactggtgc cctcgccatg |
| 6721 | agactcactg acgaggactt ggatttcctt acgaaatgga ctgattttaa gtgctttgtt |
| 6781 | tctgcgtcca acatgaggaa tgcagcgggc caatttatcg aggctgctta tgcaaaagca |
| 6841 | ctaagaattg aacttgctca gttggtacag gttgataagg tccgaggtac catggccaaa |
| 6901 | ctcgaggctt ttgccgatac cgtggcaccc caactctcgc ccggtgacat tgttgttgcc |
| 6961 | cttggccaca cgcctgttgg cagcatcttc gacctaaagg ttggtagcac caagcatact |
| 7021 | ctccaagcca ttgagactag agtccttgcc gggtccaaaa tgactgtggc gcgtgtcgtt |
| 7081 | gacccaaccc ccgcaccccc acccgtacct gtgcccatcc ctctcccacc gaaagttctg |
| 7141 | gagaacggtc ccaatgcctg gggggatgag gaccgtttga acaagaagaa gaggcgcagg |
| 7201 | atggaagccg tcggcatttt tgtcatggac gggaagaagt accagaaatt ttgggacaag |
| 7261 | aattccggtg atgtgtttta tgaggaggtc catattagca cagacgagtg ggagtgcctt |
| 7321 | agaactggcg accctgtcga ctttgatcct gagacaggga ttcagtgtgg gcatatcacc |
| 7381 | attgaagaca aggtttacaa tgtcttcacc tccccatctg gtaggagatt cttggtcccc |
| 7441 | gccaaccccg agaatagaag agctcagtgg gaagccgcca agctttccgt ggagcaagcc |
| 7501 | cttggcatga tgaacgtcga cggcgaactg actgccaaag aactggagaa actgaaaaga |
| 7561 | ataattgaca aactccaggg cctgactaag gagcagtgtt taaactgcta gccgccagcg |
| 7621 | gcttgacccg ctgtggtcgc ggcggcttag ttgttactga gacagcggta aaaatagtca |
| 7681 | aatttcacaa ccggaccttc accctaggac ctgtgaactt aaaagtggcc agtgaggttg |
| 7741 | agctaaaaga cgcggttgag cacaaccaac atccggttgc cagaccggtt gatggtggtg |
| 7801 | ttgtgctcct gcgctctgca gttccttcgc ttatagatgt cttgatctcc ggcgctgatg |
| 7861 | catctcctaa gttactcgcc cgccacgggc cgggaaacac tgggattgat ggcacgcttt |
| 7921 | gggattttga ggccgaggct actaaagagg aagttgcact cagtgcgcaa ataatacagg |
| 7981 | cttgtgatat taggcgcggc gacgcgcctg aaattggtct cccttataag ttgtaccctg |
| 8041 | ttaggggcaa ccctgagcgg gtaaaaggag ttttacagaa tacaaggttt ggagacatac |
| 8101 | cttacaaaac ccccagtgac actggaagcc cggtgcacgc ggctgcctgc ctcacgccta |
| 8161 | atgctactcc ggtgactgat gggcgctccg tcttggctac aaccatgccc tctggctttg |
| 8221 | agttgtatgt gccgaccatt ccagcgtccg tccttgatta tcttgattct aggcctgact |
| 8281 | gccctaaaca gttaacagag cacggttgtg aggatgctgc attaagagac ctctccaagt |
| 8341 | atgatttgtc cacccaaggc tttgttttgc ctggagttct tcgcctcgtg cggaagtacc |
| 8401 | tgttcgccca cgtgggtaag tgcccgcccg ttcatcggcc ttccacttac cctgctaaga |
| 8461 | attctatggc tggaataaat gggaacaggt ttccaaccaa ggacattcag agcgtccctg |
| 8521 | aaatcgacgt tctgtgcgca caggctgtgc gagaaaactg gcaaactgtt accccttgta |
| 8581 | ccctcaagaa acagtactgt gggaagaaga agactaggac aatacttggc accaataact |
| 8641 | tcattgcgtt ggcccatcgg gcagcgttga gtggtgttac ccagggcttc atgaaaaaag |
| 8701 | cgttcaactc gcccatcgcc ctcgggaaaa acaaatttaa ggagctacaa gccccggtcc |
| 8761 | taggcaggtg ccttgaagct gatcttgcgt cctgcgatcg atccacacct gcaattgtcc |
| 8821 | gctggtttgc cgccaatctt ctttatgaac tcgcctgtgc tgaggagcat ctaccgtcgt |
| 8881 | acgtgctgaa ctgctgccac gacttactgg tcacgcagtc cggcgcggtg actaagaagg |
| 8941 | gtggcctgtc gtctggcgac ccgattacct ctgtgtcaaa caccatttac agcttagtga |
| 9001 | tatatgcaca gcacatggtg ctcagttact tcaaaagtgg tcaccctcat ggccttctgt |
| 9061 | ttctgcaaga ccagctaaag tttgaggaca tgctcaaggt tcaacccctg atcgtctatt |
| 9121 | cggacgacct tgtgctgtat gccgagtctc cctccatgcc aaactaccac tggtgggttg |
| 9181 | aacatctgaa tcttatgctg ggtttccaga cggacccaaa gaagacaacc atcacagact |
| 9241 | caccatcatt cctaggttgc aggataataa atgggcgcca gctagtccct aaccgtgaca |
| 9301 | ggatcctcgc ggccctcgcc taccacatga aggcaagtaa tgtttctgaa tactacgcct |
| 9361 | cggcggctgc aatactcatg gacagctgtg cttgtttaga gtatgatcct gaatggtttg |
| 9421 | aagagctcgt ggttgggatg gcgcagtgcg cccgcaagga cggctacagc tttcctggcc |
| 9481 | caccgttctt cttgtccatg tgggaaaaac tcaggtccaa tcatgagggg aagaagtcca |
| 9541 | gaatgtgcgg gtactgcggg gccccggctc cgtacgccac tgcctgtggt ctcgatgtct |
| 9601 | gtgtttacca cacccacttc caccagcatt gtcctgttat aatctggtgt ggccacccgg |
| 9661 | cgggttctgg ttcttgtagt gagtgcgaac cccccctagg aaaaggcaca agccctctag |
| 9721 | atgaggtgtt agaacaagtt ccgtacaagc ctccgcggac tgtgatcatg catgtggagc |
| 9781 | agggtctcac ccctcttgac ccaggtagat accagactcg ccgcggatta gtctccgtta |
| 9841 | ggcgtggcat taggggaaat gaagtcgacc taccagacgg tgattacgct agcaccgcct |
| 9901 | tgctccctac ttgtaaagag atcaacatgg tcgctgtcgc ctctaacgtg ttgcgcagca |
| 9961 | ggtttatcat cggcccaccc ggtgctggga aaacacactg gcttcttcaa caagtccagg |
| 10021 | atggtgatgt catttacacg ccaactcacc agaccatgct cgacatgatt agggctttgg |
| 10081 | ggacgtgccg gttcaacgtt ccagcaggta caacgctgca attccctgcc ccctcccgta |
| 10141 | ccggcccatg ggttcgcatc ttggccggcg gttggtgtcc tggcaagaac tccttcctgg |
| 10201 | atgaagcggc gtattgcaat caccttgatg tcttgaggct tctcagtaaa acaactctca |
| 10261 | cttgcctagg agacttcaaa caactccacc ctgtgggttt tgactcccat tgctatgtat |
| 10321 | ttgacatcat gcctcagacc caattaaaga ccatctggag gttcgggcag aatatctgtg |
| 10381 | atgccattca accagattac agggacaaac ttatgtccat ggtcaacacg acccgtgtga |
| 10441 | cctacgtgga aaaacctgtc aggtatgggc aagtcctcac cccctaccac agggaccgag |
| 10501 | aggacggcgc cattactatc gactccagtc aaggcgccac atttgatgtg gttacactgc |
| 10561 | atttgcccac taaagattca ctcaacaggc aaagagctct tgttgctatc accagggcaa |
| 10621 | gacatgctat cttcgtgtat gacccacaca ggcaattgca gagcatgttt gatcttcccg |
| 10681 | cgaaaggcac acccgtcaac ctcgcagtgc accgtgacga acagctgatc gtattagaca |
| 10741 | gaaacaacag agaaatcacg gttgctcagg ctctaggcaa tggagataaa ttcagggcca |
| 10801 | cagataagcg cgttgtagat tctctccgcg ctatttgcgc agacctggaa gggtcgagct |
| 10861 | ccccgctccc caaggtcgcg cataacttgg gattctattt ctcacctgat ttgactcagt |
| 10921 | ttgctaaact cccggcagaa cttgcgcccc actggcccgt ggtgacaacc cagaacaatg |
| 10981 | aaaggtggcc agatcggctg gtagccagcc ttcgccctat ccataaatat agccgcgcgt |
| 11041 | gcattggtgc cggctatatg gtgggcccct cggtgttttt aggcacccct ggggttgtgt |
| 11101 | catactatct cacaaaattt gttagaggcg aggctcaagt gcttccggag acagtcttca |
| 11161 | gcaccggccg aattgaggta gattgtcgag agtatcttga tgatcgggag cgagaagttg |
| 11221 | ctgagtccct cccacatgcc ttcatcggcg atgtcaaagg taccaccgtt gggggatgtc |
| 11281 | atcacgttac ctccaaatac cttccgcgct tccttcccaa ggaatcagtt gcggtggtcg |
| 11341 | gggtttcgag ccccgggaaa gccgcgaaag cagtttgcac attgacggat gtgtacctcc |
| 11401 | cagaccttga agcgtacctc cacccagaga cccagtccag gtgctggaaa gtgatgttgg |
| 11461 | actttaagga ggttcgactg atggtatgga aagacaagac ggcctatttt caacttgaag |
| 11521 | gccgccattt tacctggtat caacttgcaa gctacgcctc atacatccga gttcctgtta |
| 11581 | attctactgt gtacttggac ccctgcatgg gccctgctct ttgcaacaga agggttgtcg |
| 11641 | ggtccaccca ttggggagct gacctcgcag tcacccctta tgattacggt gccaaaatta |
| 11701 | ttctgtctag tgcataccat ggtgaaatgc ctccaggtta caaaattctg gcgtgcgcgg |
| 11761 | agttctcgct tgatgaccca gtaaggtaca aacacacctg gggatttgaa tcggatacag |
| 11821 | cgtatctgta cgagtttact ggaaatggtg aggactggga ggattacaat gatgcgtttc |
| 11881 | gggcgcgcca gaaagggaaa atttataaag ctaatgccac cagcatgagg tttcattttc |
| 11941 | ccccgggccc tgtcattgaa ccaactttag gcctgaattg aaatgaaatg gggtctatgc |
| 12001 | aaagcctctt tgacaaaatt ggccaacttt ttgtggatgc tttcacggaa tttctggtgt |
| 12061 | ccattgttga tatcatcata tttttggcca ttttgtttgg cttcacaatc gccggttggc |
| 12121 | tggtggtctt atgcatcaga ctggtttgct ccgcggtact ccgtgcgcgc tctaccgttc |
| 12181 | accctgagca attacagaag atcttatgag gcctttcttt ctcagtgtca ggtggacatt |
| 12241 | cccacctggg gcgtcaaaca ccctttgggg gtgctttggc accataaggt gtcaaccctg |
| 12301 | attgatgaaa tggtgtcgcg tcgaatgtac cgcatcatgg aaaaagcagg gcaggctgcc |
| 12361 | tggaaacagg tggtgagcga ggctacattg tctcgcatta gtggtttgga tgtggtggct |
| 12421 | cactttcaac atcttgccgc tattgaagcc gagacttgta aatatttggc ctcccggcta |
| 12481 | cccatgctgc acaacctgcg cttgacaggg tcaaatgtaa ccatagtgta taatagtact |
| 12541 | ttggatcagg tgtttgccat tttcccaacc cctggttccc ggccaaagct tcatgatttt |
| 12601 | cagcaatggc taatagctgt acattcctcc atattttctt ccgttgcagc ttcttgtact |
| 12661 | ctttttgttg tgctgtggtt gcgaattcca atgctacgtt ctgtttttgg tttccgctgg |
| 12721 | ttaggggcaa cttttctttt gaactcatgg tgaattacac ggtatgcccg ctttgcccaa |
| 12781 | cccggcaggc agccgctgag atccttgaac ccggcaagtc tttttggtgc aggatagggc |
| 12841 | atgaccgatg tagtgagaac gatcatgacg aactagggtt catggttccg cctggccttt |
| 12901 | ccagcgaagg ccacttgacc agtgtttacg cctggttggc gttcctgtcc ttcagctaca |
| 12961 | cggcccagtt ccatcccgag atatttggga tagggaatgt gagtcaagtt tatgttgaca |
| 13021 | tcaagcacca attcatctgc gctgttcacg acggggataa cgccaccttg cctcgccatg |
| 13081 | acaatatttc agccgtattt cagacctact accaacacca ggtcgacggc ggcaattggt |
| 13141 | ttcacctgga atggctgcgc cctttctttt cctcttggtt ggttttaaat gtttcgtggt |
| 13201 | ttctcaggcg ttcgcctgca aaccatgttt cagttcgagt ctttcggaca tcaaaaccaa |
| 13261 | caccaccgca gcatcagact tcgttgtcct ccaggacatc agctgcctta ggcatggcga |
| 13321 | ctcgtcctct ccgacgattc gcaaaagttc tcagtgccgc acggcgatag ggacgcccgt |
| 13381 | gtacatcacc atcactgcca atgtcacaga tgaaaattat ctacattctt ctgatctcct |
| 13441 | catgctttct tcttgccttt tctatgcttc cgagatgagt gaaaagggat tcaaagtggt |
| 13501 | gtttggcaat gtgtcaggca tcgtggctgt gtgcgtcaac tttaccagct acgtccaaca |
| 13561 | cgtcaaggag tttacccaac gctccttagt ggtcgatcat gtgcgactgc ttcatttcat |
| 13621 | gacacctgag accatgaggt gggcaaccgt tttagcctgt ctttttgcca tcctactggc |
| 13681 | aatttgaatg tttaagtatg ttggggaagt gcttgaccgc gtgctgttgc tcgcgattgc |
| 13741 | tttttttgtg gtgtatcgtg ccgttctatc ttgctgtgct cgtcaacgcc agcaacaaca |
| 13801 | acagctctca tattcagttg atttataact taacgctatg tgagctgaat ggcacagatt |
| 13861 | ggctggcaca aaaatttgac tgggcagtgg agacttttgt catcttcccc gtgttgactc |
| 13921 | acattgtttc ctatggggca ctcaccacca gccatttcct tgacacagtt ggtctggcca |
| 13981 | ctgtgtccac cgccggatat tatcacgggc ggtatgtctt gagtagcatt tacgcagtct |
| 14041 | gtgctctggc tgcgctgatt tgctttgtca ttaggcttgc gaagaactgc atgtcctggc |
| 14101 | gctactcttg taccagatat accaacttcc ttctggacac taagggcaga ctctatcgtt |
| 14161 | ggcggtcgcc cgtcattgtg gagaaagggg gtaaggttga ggtcgaaggt cacctgatcg |
| 14221 | acctcaagag agttgtgctt gatggttccg cggcaacccc tttaaccaga gtttcagcgg |
| 14281 | aacaatgggg tcgtctctag acgacttctg caatgatagc acagctccac agaaggtgct |
| 14341 | tttggcgttt tccattacct acacgccagt gatgatatat gctctaaagg taagtcgcgg |
| 14401 | ccgactgcta gggcttctgc accttttgat ctttctgaat tgtgctttta ccttcgggta |
| 14461 | catgacattc gtgcactttg agagcacaaa tagggtcgcg ctcactatgg gagcagtagt |
| 14521 | tgcacttctt tggggagtgt actcagccat agaaacctgg aaattcatca cctccagatg |
| 14581 | ccgtttgtgc ttgctaggcc gcaagtacat tctggcccct gcccaccacg tcgaaagtgc |
| 14641 | cgcgggcttt catccgattg cggcaaatga taaccacgca tttgtcgtcc ggcgtcccgg |
| 14701 | ctccactacg gtcaacggca cattggtgcc cgggttgaaa agcctcgtgt tgggtggcag |
| 14761 | aaaagctgtt aagcagggag tggtaaacct tgttaaatat gccaaataac aacggcaagc |
| 14821 | agcaaaagaa aaagaagggg aatggccagc cagtcaatca gctgtgccaa atgctgggta |
| 14881 | agatcatcgc ccaacaaaac cagtccagag gcaagggacc ggggaagaaa aataggaaga |
| 14941 | aaaacccgga gaagccccat ttccctctag cgactgaaga tgacgtcagg catcacttta |
| 15001 | cccctagtga gcggcaattg tgtctgtcgt cgatccagac tgccttcaat cagggcgctg |
| 15061 | gaacttgtgc cctgtcagat tcagggagga taagttacac tgtggagttt agtttgccga |
| 15121 | cgcaacatac tgtgcgtctg atccgcgcca cagcatcacc ctcagcatga tgggctggca |
| 15181 | ttctttggca cctcagtgtt agaattggga gaatgtgtgg tgaatggcac tgattgacac |
| 15241 | tgtgcctcta agtcacctat tcaattaggg cgaccgtgtg ggggtaaagt ttaattggcg |
| 15301 | agaaccatgc ggccgtaatt aaaaaaaaaa aaaaaaaaaa aaaaaaaaaa aaaaaaaaaa |
| 15361 | aaaaaaaaaa aaa |
| SEQ ID NO: 2 | |
| (NSP2 of VR2332, SwissProt accession No. Q9WJB2) | |
| 1 | GAGKRARKAR SCATATVAGR ALSVRETRQA KEHEVAGANK AEHLKHYSPP AEGNCGWHCI |
| 61 | SAIANRMVNS KFETTLPERV RPPDDWATDE DLVNAIQILR LPAALDRNGA CTSAKYVLKL |
| 121 | EGEHWTVTVT PGMSPSLLPL ECVQGCCGHK GGLGSPDAVE VSGFDPACLD RLAEVMHLPS |
| 181 | SAIPAALAEM SGDSDRSASP VTTVWTVSQF FARHSGGNHP DQVRLGKIIS LCQVIEDCCC |
| 241 | SQNKTNRVTP EEVAAKIDLY LRGATNLEEC LARLEKARPP RVIDTSFDWD VVLPGVEAAT |
| 301 | QTIKLPQVNQ CRALVPVVTQ KSLDNNSVPL TAFSLANYYY RAQGDEVRHR ERLTAVLSKL |
| 361 | EKVVREEYGL MPTEPGPRPT LPRGLDELKD QMEEDLLKLA NAQTTSDMMA WAVEQVDLKT |
| 421 | WVKNYPRWTP PPPPPKVQPR KTKPVKSLPE RKPVPAPRRK VGSDCGSPVS LGGDVPNSWE |
| 481 | DLAVSSPFDL PTPPEPATPS SELVIVSSPQ CIFRPATPLS EPAPIPAPRG TVSRPVTPLS |
| 541 | EPIPVPAPRR KFQQVKRLSS AAAIPPYQDE PLDLSASSQT EYEASPPAPP QSGGVLGVEG |
| 601 | HEAEETLSEI SDMSGNIKPA SVSSSSSLSS VRITRPKYSA QAIIDSGGPC SGHLQEVKET |
| 661 | CLSVMREACD ATKLDDPATQ EWLSRMWDRV DMLTWRNTSV YQAICTLDGR LKFLPKMILE |
| 721 | TPPPYPCEFV MMPHTPAPSV GAESDLTIGS VATEDVPRIL EKIENVGEMA NQGPLAFSED |
| 781 | KPVDDQLVND PRISSRRPDE STSAPSAGTG GAGSFTDLPP SDGADADGGG PFRTVKRKAE |
| 841 | RLFDQLSRQV FDLVSHLPVF FSRLFYPGGG YSPGDWGFAA FTLLCLFLCY SYPAFGIAPL |
| 901 | LGVFSGSSRR VRMGVFGCWL AFAVGLFKPV SDPVGAACEF DSPECRNILH SFELLKPWDP |
| 961 | VRSLVVGPVG LGLAILGRLL GGARCIWHFL LRLGIVADCI LAGAYVLSQG RCKKCWGSCI |
| 1021 | RTAPNEVAFN VFPFTRATRS SLIDLCDRFC APKGMDPIFL ATGWRGCWAG RSPIEQPSEK |
| 1081 | PIAFAQLDEK KITARTVVAQ PYDPNQAVKC LRVLQSGGAM VAKAVPKVVK VSAVPFRAPF |
| 1141 | FPTGVKVDPD CRVVVDPDTF TAALRSGYST TNLVLGVGDF AQLNGLKIRQ ISKPSG |
| SEQ ID NO: 3 | |
| ORF5 SEQUENCE OF PRRS VR2332 (GENBANK ACCESSION NO. U87392) | |
| 1 | atgttggaga aatgcttgac cgcgggctgt tgctcgcgat tgctttcttt gtggtgtatc |
| 61 | gtgccgttct gttttgctgt gctcgccaac gccagcaacg acagcagctc ccatctacag |
| 121 | ctgatttaca acttgacgct atgtgagctg aatggcacag attggctagc taacaaattt |
| 181 | gattgggcag tggagagttt tgtcatcttt cccgttttga ctcacattgt ctcctatggt |
| 241 | gccctcacta ccagccattt ccttgacaca gtcgctttag tcactgtgtc taccgccggg |
| 301 | tttgttcacg ggcggtatgt cctaagtagc atctacgcgg tctgtgccct ggctgcgttg |
| 361 | acttgcttcg tcattaggtt tgcaaagaat tgcatgtcct ggcgctacgc gtgtaccaga |
| 421 | tataccaact ttcttctgga cactaagggc agactctatc gttggcggtc gcctgtcatc |
| 481 | atagagaaaa ggggcaaagt tgaggtcgaa ggtcatctga tcgacctcaa aagagttgtg |
| 541 | cttgatggtt ccgtggcaac ccctataacc agagtttcag cggaacaatg gggtcgtcct |
| 601 | tag |

1. Method of vaccinating swine against the effects of a high fever disease form of PRRS, comprising administering to a pig an immunogenic composition comprising an effective amount of a PRRS Type II virus.

2. Method of claim 1, wherein said high fever disease form of PRRS is from a Chinese PRRSV that has a nucleic acid sequence that is at least 95% homologous to the nucleic acid sequence of HB-1, or JX143.

3. Method of claim 1 wherein the high fever disease form is caused by a HP PRRS virus.

4. Method of claim 1, wherein said PRRS type II virus is attenuated.

5. Method of claim 3, characterized in that the PRRS type II virus is an attenuated form of the strain with the accession No. ATCC VR-2332, or a descendant thereof.

6. Method of claim 4, characterized in that the PRRS type II virus is a strain with the accession No. ATCC VR-2495, or a descendant thereof.

7. Method of claim 1 wherein said Chinese PRRSV strain is selected from the group consisting of AH-1; AHCFSH; AHCFZC; BB07; BD-8; BQ07; CL07; CX07; CZ07; FY060915; FY080108; GC-2; GCH-3; GD1; GD2; GD2007; GD3; GD4; GDSD1; GDY1-2007; GDY2-2007; GDYF1; GS2008; GXHZ12; GXHZ13; GXHZ14; GXHZ16; GXHZ19; GXHZ2; GXHZ21; GXHZ4; GXLZ5; GXLZ7; GY; GZCJ; GZDJ; GZHW1; GZHW2; GZHX; GZJS; GZKB; GZKY; GZLJ1; GZWB; GZWM; GZZB; Hainan-1; Hainan-2; HB1; HB2; HB3; HB-Tsh1; HB-Xt1; HEN46; HeN-KF; HeN-LH; HeN-LY; HLJDF; HLJMZ1; HLJMZ2; HLJMZ3; HLJZY; HM-1; HN2; HN2007; HN3; HNId; HNIy; HNLY01; HNNX01; HNPJ01; HNsp; HNXT1; HNyy; HNyz; HQ-5; HQ-6; HUB; HuN; HUN1; HUN11; HUN15; HUN16; HUN17; HUN2; HUN3; HUN4; HUN5; HUN6; HUN7; Hunan-1; Hunan-2; Hunan-3; HUNH2; HUNH4; HuNhl; HUNL1; HUNX4; HZ061226; HZ070105; Jiangsu-1; Jiangsu-2; Jiangsu-3; Jiangxi-2; Jiangxi-4; JLYS; JN; JX1; JX143; JX2; JX-2; JX2006; JX3; JX4; JX5; JXA1; KS06; LC07; LJ; LS06; LS-4; LY07; NB070319; SC07; SD; SD14; SDWF2; SH02; ST-7; SX2007; SY0608; TJDMJ; TJZHJ2; TJZHJ3; TQ; TQ07; TWO7; WF07; XJ07; XL2008; YN2008; YNBS; YNDL; YNMG; YNWS; YNYS; YNYX1; YNYX3; ZJ06; ZJCJ; ZJWL; ZX07; and ZS070921

8. Method of any one of claim 1 to 7 or 9, 10 or 11, wherein the composition further comprises an adjuvant.

9. Method of vaccinating swine against the effects of a high fever disease form of PRRS virus JX143, comprising administering to a pig an immunogenic composition comprising an effective amount of a PRRS Type II virus.

10. Method of lessening of the incidence of or severity of clinical signs of high fever disease forms of PRRS, comprising administering to a pig in need thereof an immunogenic composition comprising an effective amount of a PRRS Type II virus.

11. Method of claim 10 wherein said high fever disease form of PRRS is from a Chinese PRRSV that has a nucleic acid sequence that is at least 95% homologous to the nucleic acid sequence of HB-1, or JX143.

12-15. (canceled)
